(19) United States
(12) Patent Application Publication
(10) Pub. No.: US 2026/0151329 A1
(43) Pub. Date: Jun. 4, 2026

(54) METHODS OF SCAR PREVENTION AND/OR TREATMENT

(71) Applicant: Allergan Pharmaceuticals International Limited, Dublin 17 (IE)

(72) Inventors: Robert DANIELS, Sydney (AU); Damien BATES, East Melbourne (AU); John St. Clair ROBERTS, Suffolk (GB)

(21) Appl. No.: 19/352,348

(22) Filed: Oct. 7, 2025

Related U.S. Application Data
(63) Continuation of application No. 18/393,925, filed on Dec. 22, 2023, now abandoned.
Application No. 18/393,925 is a continuation of application No. 16/865,309, filed on May 2, 2020, now abandoned.
(60) Provisional application No. 62/843,302, filed on May 3, 2019.

(51) Int. Cl. A61K 9/00; A61K 31/728; A61K 38/39; A61K 47/61; A61K 47/64; A61P 17/02

(52) U.S. Cl. CPC A61K 9/0014; A61K 31/728; A61K 38/39; A61K 47/61; A61K 47/6435; A61P 17/02

(57) ABSTRACT

The disclosure relates to methods of treating and/or preventing scarring that would otherwise result from an incision to an area of skin. The methods comprise administering a composition comprising tropoelastin to an area of skin that is to receive an incision and/or after the incision. It is also contemplated that the compositions may reduce scar discoloration. Methods of preventing or reducing keloid re-occurrence are also provided.

### CROSS REFERENCE TO RELATED APPLICATIONS

This Application is a continuation of U.S. patent application Ser. No. 18/393,925 filed Dec. 22, 2023, which is a continuation of U.S. patent application Ser. No. 16/865,309 filed May 2, 2020, which claims the benefit of and priority to U.S. Provisional Patent Application No. 62/843,302 filed May 3, 2019, which are hereby incorporated by reference in their entireties.

### REFERENCE TO SEQUENCE LISTING SUBMITTED ELECTRONICALLY

A copy of the Sequence Listing is being submitted in computer readable form (CRF) in accordance with the requirements of 37 C.F.R. §§ 1.821-1.825. The sequence listing is submitted electronically via Patent Center as an XML formatted sequence listing with a file named 051749-317C02US_SL_ST26.xml created on Oct. 7, 2025, and having a size of 25,120 bytes. The sequence listing contained in this XML formatted document is part of the specification and is herein incorporated by reference in its entirety.

### BACKGROUND

### Field of the Disclosure

The disclosure relates to methods of treating and/or preventing the formation of a scar in a subject in need thereof. These methods include administering a composition comprising tropoelastin to an area of skin prior to and/or after a surgical procedure that introduces an incision to the area of skin.

### Description of the Related Art

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

Healing of a tissue may include processes such as repairing or regenerating itself after injury. Some tissues are capable of regeneration but may tend towards a repair mechanism involving fibrosis if appropriate conditions are not found. Skin is one example.

The classic model of wound healing may be divided into three or four sequential, yet overlapping, phases: (1) hemostasis, (2) inflammatory, (3) proliferative and (4) remodeling. The proliferative phase is characterized by angiogenesis, collagen deposition, granulation tissue formation and re-epithelialization.

Angiogenesis occurs at the same time as fibroblast proliferation. Angiogenesis is imperative for other stages in wound healing because the activity of fibroblasts and epithelial cells requires oxygen and nutrients. According to the process, stem cells of endothelial cells and other vascular cells originating from the circulation and parts of uninjured blood vessels, develop pseudopodia and push through the extracellular matrix (ECM) into the wound site to establish new blood vessels. Endothelial cells are attracted to the wound area by fibronectin found on the fibrin scab and chemotactically by angiogenic factors released by other cells, e.g. from macrophages and platelets when in a low oxygen environment. To migrate, endothelial cells need collagenases and plasminogen activator to degrade the clot and part of the ECM. Zinc-dependent metalloproteinases digest basement membrane and ECM to allow cell migration, proliferation and angiogenesis. When tissue is adequately perfused, migration and proliferation of endothelial cells is reduced. Eventually blood vessels that are no longer needed die by apoptosis.

Collagen production and deposition is important because it increases the strength of the wound by providing more resistance to force than a fibrin-fibronectin clot. Also, cells involved in inflammation, angiogenesis, and connective tissue construction attach to, grow and differentiate on the collagen matrix laid down by fibroblasts. Type III collagen and fibronectin are generally beginning to be produced in appreciable amounts at somewhere between approximately 10 hours and 3 days, depending mainly on wound size. Their deposition peaks at one to three weeks. They are the predominating tensile substances until the later phase of maturation, in which they are replaced by the stronger type I collagen. Even as fibroblasts are producing new collagen, collagenases and other factors degrade it. Shortly after wounding, synthesis exceeds degradation so collagen levels in the wound rise, but later production and degradation become equal so there is no net collagen gain. This homeostasis signals the onset of the later maturation phase.

In the first two or three days after injury, fibroblasts mainly migrate and proliferate, while later, as described above, they are the main cells that lay down the collagen matrix in the wound site. Origins of these fibroblasts are thought to be from the adjacent uninjured cutaneous tissue. Initially, fibroblasts utilize the fibrin cross-linking fibers that are formed by the end of the inflammatory phase to migrate across the wound, subsequently adhering to fibronectin. Fibroblasts then deposit ground substance into the wound bed, and later collagen, which they can adhere to for migration, thereby producing the basis for formation of granulation tissue.

Granulation tissue functions as rudimentary tissue and begins to appear in the wound already during the inflammatory phase, two to five days post wounding, and continues growing until the wound bed is covered. Granulation tissue consists of new blood vessels, fibroblasts, inflammatory cells, endothelial cells, myofibroblasts, and the components of a new, provisional ECM. The provisional ECM is different in composition from the ECM in normal tissue and its components originate from fibroblasts. Such components include fibronectin, collagen, glycosaminoglycans, elastin, glycoproteins and proteoglycans. The main components of the provisional ECM are fibronectin and hyaluronan, which create a very hydrated matrix and facilitate cell migration. Later this provisional matrix is replaced with an ECM that more closely resembles that found in non-injured tissue. At the end of the granulation phase, fibroblasts undergo apoptosis, converting granulation tissue from an environment rich in cells to one that consists mainly of collagen.

The result of wound healing in the skin almost always results in the formation of a visible scar, which may appear red or inflamed initially, and will take up to 12 months to fully mature. The ideal outcome is for the scar to be narrow, and level with the surrounding skin so as to be minimally perceptible; however, different injuries and damage to the skin creates wounds of varying sizes and different people heal differently, resulting in a wide range of scar severities.

Scar tissue is mainly comprised of the fibrous protein collagen that is formed during the process of wound repair as described above and is a natural part of healing. However, some degree of visible scarring will almost always result from the wound repair process. Without being limiting, visible scarring may occur from medical and cosmetic surgeries, for example, C-sections, laparotomies, open heart surgeries, surgical removal of tumours or cancers, breast reduction, breast augmentation, reconstructive procedures, accidental lacerations, and dermatological procedures, including excisions of scars, such as a keloid, excisions of melanocytic nevi, excisions of potentially cancerous tissue, such as basal cell carcinoma and melanoma, and other procedures.

Even when appropriate measures are taken to allow an incision to heal properly, a noticeable scar may still form. Indeed, during a surgical procedure, scar tissue formation and contracture may be a problem as they may have unsightly aesthetic effects to a patient. As such, there is a need to reduce and prevent unsightly scar tissue from forming.

### SUMMARY

The present disclosure provides methods of treating or preventing scarring that would otherwise result from an incision to an area of skin by administering a composition comprising tropoelastin to the area of skin. Such administration may be made prior to and/or after an incision is made to the area of skin.

In a first aspect, the disclosure provides methods of preventing scarring from an incision to an area of skin including, for example, by pre-treating the area of skin of a subject in need thereof to prevent scarring that would otherwise result from making an incision to the area of skin.

In some embodiments of each or any of the above- or below-mentioned embodiments, the methods may further comprise making an incision to the area of skin.

In some embodiments of each or any of the above- or below-mentioned embodiments, the methods comprise administering a composition comprising tropoelastin to an area of skin that is to receive an incision (e.g., administering the composition comprising tropoelastin to the area of skin as a pre-treatment step prior to making an incision to that area of skin).

In some embodiments of each or any of the above- or below-mentioned embodiments, the methods comprise administering a composition comprising tropoelastin to an area of skin comprising a proposed incision site, wherein the administering is performed along a length of the proposed incision site about 1 minute to about 90 days prior to the proposed incision site receiving an incision.

In some embodiments of each or any of the above- or below-mentioned embodiments, the administering is performed about every 0.1 cm, 0.2 cm, 0.3 cm, 0.4 cm, 0.5 cm, 0.6 cm, 0.7 cm, 0.8 cm, 0.9 cm or 1.0 cm along the length of the proposed incision site.

In some embodiments of each or any of the above- or below-mentioned embodiments, the proposed incision site has a right side and a left side, wherein the composition is administered along the right side and/or the left side along the length of the proposed incision site, and wherein the composition is injected about 0.1 cm, about 0.2 cm, about 0.3 cm, about 0.4 cm, about 0.5 cm, about 0.6 cm, about 0.7 cm, about 0.8 cm about 0.9 cm, about 1 cm, about 1.1 cm, about 1.2 cm, about 1.3 cm, about 1.4 cm or about 1.5 cm away from the proposed incision site.

In some embodiments of each or any of the above- or below-mentioned embodiments, the incision site comprises a first end and a second end, wherein the composition is administered along the first end and/or the second end of the proposed incision site, and wherein the composition is injected about 0.1 cm, about 0.2 cm, about 0.3 cm, about 0.4 cm, about 0.5 cm, about 0.6 cm, about 0.7 cm, about 0.8 cm about 0.9 cm, about 1 cm, about 1.1 cm, about 1.2 cm, about 1.3 cm, about 1.4 cm or about 1.5 cm away from the proposed incision site at the first end and/or the second end.

In some embodiments of each or any of the above- or below-mentioned embodiments, the tropoelastin is a recombinant human tropoelastin.

In some embodiments of each or any of the above- or below-mentioned embodiments, the composition comprises between about 1-100 mg/ml of a tropoelastin. In further embodiments, the composition comprises about 10 mg/ml, about 20 mg/ml, about 30 mg/ml, about 40 mg/ml or about 50 mg/ml of a recombinant human tropoelastin.

In some embodiments of each or any of the above- or below-mentioned embodiments, the composition further comprises hyaluronic acid. In some embodiments of each or any of the above- or below-mentioned embodiments, the hyaluronic acid comprises derivatized hyaluronic acid (dHA). In some embodiments of each or any of the above- or below-mentioned embodiments, the composition comprises hyaluronic acid in a concentration of about 0.2% to about 10% hyaluronic acid. In some embodiments of each or any of the above- or below-mentioned embodiments, the composition comprises about 0.2%, about 0.3%, about 0.4%, about 0.5%, about 0.6%, about 0.7%, about 0.8%, 0.9% or about 1% derivatized hyaluronic acid.

In some embodiments of each or any of the above- or below-mentioned embodiments, the tropoelastin is in a monomeric form.

In some embodiments of each or any of the above- or below-mentioned embodiments, the tropoelastin is cross-linked to the hyaluronic acid. In some embodiments of each or any of the above- or below-mentioned embodiments, the tropoelastin is cross-linked to the derivatized hyaluronic acid.

In some embodiments of each or any of the above- or below-mentioned embodiments, the composition comprises about 30 mg/ml tropoelastin.

In some embodiments of each or any of the above- or below-mentioned embodiments, the tropoelastin comprises about 30 mg/ml tropoelastin crosslinked to about 0.5% derivatized hyaluronic acid.

In some embodiments of each or any of the above- or below-mentioned embodiments, the composition further comprises a buffer, wherein the buffer is phosphate buffered saline (PBS).

In some embodiments of each or any of the above- or below-mentioned embodiments, the composition is administered along the length of the proposed incision site. In some embodiments of each or any of the above- or below-mentioned embodiments, the composition is administered by injection in parallel along the length of the proposed incision site, wherein each injection is equidistant from the proposed incision site. In some embodiments of each or any of the above- or below-mentioned embodiments, the composition is administered by injection along the length of the proposed incision site, wherein the injections sites are not all equidistant from the proposed incision site.

In some embodiments of each or any of the above- or below-mentioned embodiments, the composition is administered in a volume of about 1 μL to about 100 μL. In some embodiments of each or any of the above- or below-mentioned embodiments, the composition is administered in a volume of about 5 μL to about 20 μL. In some embodiments of each or any of the above- or below-mentioned embodiments, the composition is administered in a volume of about 10 μL to about 30 μL. In some embodiments of each or any of the above- or below-mentioned embodiments, the composition is administered in a volume of about 20 μL to about 50 μL. In some embodiments of each or any of the above- or below-mentioned embodiments, the composition is administered in a volume of about 25-50 μL.

In some embodiments of each or any of the above- or below-mentioned embodiments, the composition is administered with a 30G needle. In some embodiments of each or any of the above- or below-mentioned embodiments, the composition is administered with a 27G needle. In some embodiments of each or any of the above- or below-mentioned embodiments, the composition is administered with a 32G, 27G, 25G, or 23G needle.

In some embodiments of each or any of the above- or below-mentioned embodiments, the composition is administered with an injection device. In some embodiments of each or any of the above- or below-mentioned embodiments, the injection device is a needle roller ball type system, an automatic injection pen type system, or a mesotherapy injection gun type system.

In some embodiments of each or any of the above- or below-mentioned embodiments, the composition is administered as an intradermal implant, a dermal implant, a subdermal implant, within a hypodermis and/or a subcutaneous area.

In some embodiments of each or any of the above- or below-mentioned embodiments, the composition is administered as an injection directly into the proposed incision site, beneath the proposed incision site, or the area of skin around the proposed incision site, along the length of the proposed incision site. In some embodiments of each or any of the above- or below-mentioned embodiments, the composition is administered as an injection directly at the proposed incision site along the length of the proposed incision site.

In some embodiments of each or any of the above- or below-mentioned embodiments, the composition is administered as an injection beneath a proposed incision site, wherein the composition is injected at about 1 mm, about 2 mm, about 3 mm, about 4 mm, about 5 mm, about 6 mm, about 7 mm, about 8 mm, about 9 mm or about 10 mm below the proposed incision site.

In some embodiments of each or any of the above- or below-mentioned embodiments, the composition is injected about 0.1 cm away from the proposed incision site, on a right side and/or a left side of the proposed incision site, wherein the composition comprises about 10 mg/ml to about 30 mg/ml recombinant human tropoelastin crosslinked with 0.5% derivatized hyaluronic acid, and wherein the composition is administered in a volume of about 1 μL to about 25 μL. In some embodiments of each or any of the above- or below-mentioned embodiments, the composition is injected about 0.5 cm away from the proposed incision site, on the right side and/or the left side of the proposed incision site, wherein the composition comprises about 30 mg/ml to about 100 mg/ml recombinant human tropoelastin crosslinked with 0.5% derivatized hyaluronic acid, and wherein the composition is administered in a volume of about 25 μL to about 50 μL. In some embodiments of each or any of the above- or below-mentioned embodiments, the composition is injected about 1.0 cm away from the proposed incision site, on the right side and/or the left side of the proposed incision site, wherein the composition comprises about 30 mg/ml to about 100 mg/ml recombinant human tropoelastin crosslinked with 0.5% derivatized hyaluronic acid, and wherein the composition is administered in a volume of about 50 μL to about 100 μL. In some embodiments of each or any of the above- or below-mentioned embodiments, the composition is injected about 1.5 cm away from the proposed incision site, on the right side and/or the left side of the proposed incision site, wherein the composition comprises about 30 mg/ml to about 100 mg/ml recombinant human tropoelastin crosslinked with 0.5% derivatized hyaluronic acid, and wherein the composition is administered in a volume of about 75 μL to about 100 μL.

In some embodiments of each or any of the above- or below-mentioned embodiments, the composition is administered by injection at about 0.7 cm to about 0.8 cm intervals along the length of the proposed incision site. In some embodiments of each or any of the above- or below-mentioned embodiments, the composition is administered by injection at 0.7 to 0.8 cm intervals along the length of the proposed incision site, on the right side and/or the left side of the proposed incision site.

In some embodiments of each or any of the above- or below-mentioned embodiments, the incision is performed for a surgical procedure.

In some embodiments of each or any of the above- or below-mentioned embodiments, the composition is administered to the area of skin comprising the proposed incision site about 90 days prior to receiving the incision. In some embodiments of each or any of the above- or below-mentioned embodiments, the composition is administered to the area of skin comprising the proposed incision site about 60 days prior to receiving the incision. In some embodiments of each or any of the above- or below-mentioned embodiments, the composition is administered to the area of skin comprising the proposed incision site about 28 days prior to receiving the incision. In some embodiments of each or any of the above- or below-mentioned embodiments, the composition is administered to the area of skin comprising the proposed incision site about 1 minute prior to receiving the incision. In some embodiments of each or any of the above- or below-mentioned embodiments, the method further comprises making an incision on the proposed incision site and administering the composition following the incision.

In some embodiments of each or any of the above- or below-mentioned embodiments, the composition is administered about 1 minute, about 5 minutes, about 10 minutes, about 20 minutes, about 30 minutes, about 1 hour, about 2 hours, about 4 hours, about 6 hours, about 12 hours, about 24 hours, about 48 hours, about 72 hours, about 1 week, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months and/or about 12 months following the incision.

In some embodiments of each or any of the above- or below-mentioned embodiments, the method further comprises repeating administration of the composition following the incision. In some embodiments of any one of each or any of the above- or below-mentioned embodiments, the composition is administered initially between about 1 minute and about 60 minutes following the incision. In some embodiments of each or any of the above- or below-mentioned embodiments, repeating administration is performed during a duration of about 1 year after making the incision.

In some embodiments of each or any of the above- or below-mentioned embodiments, the composition is administered repeatedly up to about 365 days following the incision.

In some embodiments of each or any of the above- or below-mentioned embodiments, the subject in need thereof has a skin type on a Fitzpatrick skin type scale of I, II, III, IV, V, or VI.

In some embodiments of each or any of the above- or below-mentioned embodiments, the area of skin that is to receive the incision is a facial area, back, torso or limbs of the subject in need thereof.

In some embodiments of each or any of the above- or below-mentioned embodiments, the subject is prone to keloid type scarring.

In some embodiments of each or any of the above- or below-mentioned embodiments, the surgery is for keloid removal, and the method further prevents keloid recurrence after surgical removal. In some embodiments of each or any of the above- or below-mentioned embodiments, the subject is prone to keloid type scarring and the method minimizes or prevents keloid scarring at the incision.

In some embodiments of each or any of the above- or below-mentioned embodiments, the method prevents formation of scar coloration.

In some embodiments of each or any of the above- or below-mentioned embodiments, the composition is administered by injection at about 0.1 to about 2.0 cm intervals (e.g., about 0.7 to about 0.8 cm intervals), along the area of skin that is to receive the incision, or along each side of the area of skin that is to receive the incision.

In some embodiments of each or any of the above- or below-mentioned embodiments, the method further comprises administering the composition again (e.g., a subsequent dose or doses of the composition) following the incision.

In some embodiments of each or any of the above- or below-mentioned embodiments, the composition is administered up to about 365 days following the incision. In some embodiments of each or any of the above- or below-mentioned embodiments, the composition is administered daily, every other day, every third day, or every fourth day for up to about 365 days following the incision.

In some embodiments of each or any of the above- or below-mentioned embodiments, the subject is prone to keloids and is administered the composition about 1 minute, about 10 minutes, about 20 minutes, about 30 minutes, about 40 minutes, about 50 minutes, about 60 minutes, about 2 hours, about 3 hours, about 4 hours, about 12 hours, about 24 hours, about 48 hours, about 72 hours, about 1 week, about 2 weeks, about 4 weeks, about 1 month, about 2 months, about 3 months, about 4 months, about 5 months, about 6 months, about 7 months, about 8 months, about 9 months, about 10 months, about 11 months, about 12 months, about 18 months, about 24 months, about 30 months, about 36 months, about 42 months, about 48 months, about 54 months, about 60 months, about 66 months, about 72 months, about 78 months, about 84 months, about 90 months, about 96 months, about 102 months, about 108 months, about 114 months, or about 120 months after the incision.

In some embodiments of each or any of the above- or below-mentioned embodiments, the subject is prone to keloids and is administered the composition about 1 minute, about 1 year, about 2 years, about 3 years, about 4 years, about 5 years, about 6 years, about 7 years, about 8 years, about 9 years or about 10 years after the incision. In some embodiments of each or any of the above- or below-mentioned embodiments, the subject is administered the composition repeatedly after a first administration of the composition. In some embodiments of each or any of the above- or below-mentioned embodiments, the subject is administered the composition repeatedly after a first administration of the composition for more than about 10 years.

In a second aspect, the disclosure provides methods of decreasing scar formation including, for example, pre-treating skin of a subject in need to decrease scar formation and scar discoloration. The methods comprise administering a composition comprising tropoelastin to an area of skin comprising a proposed incision site, wherein the administering is performed along a length of the proposed incision site about 1 minute to about 90 days prior to receiving an incision (e.g., administering tropoelastin to the area of skin as a pre-treatment step prior to making an incision to that area of skin). In some embodiments of each or any of the above- or below-mentioned embodiments, the method prevents red, purple, dark brown or white scarring.

In a third aspect, the disclosure provides methods of performing a surgical procedure, the method comprising administering a composition comprising tropoelastin to an area of skin comprising a proposed incision site, wherein the administering is performed about 1 minute to about 90 days along a length of the proposed incision site prior to making an incision; and making the incision to the area of skin.

In a fourth aspect, the disclosure provides methods of preventing scarring or scar discoloration in a subject in need thereof, the method comprising administering a composition comprising tropoelastin to an area of skin comprising an incision site, wherein the administering is performed along a length of the incision site about 1 minute to about 365 days after receiving the incision.

In some embodiments, the incision is from surgery.

In some embodiments of each or any of the above- or below-mentioned embodiments, sutures are needed to close the incision, and the administering of the composition is performed prior to and/or after the incision has received sutures.

In some embodiments of each or any of the above- or below-mentioned embodiments, the administering is performed along a length of the incision and the composition is administered every 0.1 cm, 0.2 cm, 0.3 cm, 0.4 cm, 0.5 cm, 0.6 cm, 0.7 cm, 0.8 cm, 0.9 cm or 1.0 cm along the length of the incision site on one or both sides of the incision. In some embodiments, the composition is injected about 0.1 cm, about 0.2 cm, about 0.3 cm, about 0.4 cm, about 0.5 cm, about 0.6 cm, about 0.7 cm, about 0.8 cm about 0.9 cm, about 1 cm, about 1.1 cm, about 1.2 cm, about 1.3 cm, about 1.4 cm or about 1.5 cm away from the incision.

In some embodiments of each or any of the above- or below-mentioned embodiments, the composition is administered by injection in parallel along the length of the proposed incision site, wherein each injection is equidistant from the proposed incision site. In some embodiments of each or any of the above- or below-mentioned embodiments, the composition is administered by injection along the length of the proposed incision site, wherein the injections sites are not all equidistant from the proposed incision site.

In a fifth aspect, a method of treating skin of a subject in need thereof to prevent scar formation or scar coloration is provided, the method comprising administering a composition comprising tropoelastin to an area of skin that has received a fresh wound, wherein the fresh wound is an incision or abrasion.

In some embodiments of each or any of the above- or below-mentioned embodiments, the fresh wound is an abrasion and the composition is administered on an area of skin around the abrasion, wherein the administering is performed around a circumference of the wound at about 0.7 cm to about 0.8 cm intervals along the circumference of the wound, and about 0.1 cm, about 0.2 cm, about 0.3 cm, about 0.4 cm, about 0.5 cm, about 0.6 cm, about 0.7 cm, about 0.8 cm about 0.9 cm, about 1 cm, about 1.1 cm, about 1.2 cm, about 1.3 cm, about 1.4 cm or about 1.5 cm away from the wound.

In some embodiments of each or any of the above- or below-mentioned embodiments, the administering is performed about 1 minute, about 5 minutes, about 10 minutes, about 20 minutes, about 30 minutes, about 60 minutes, about 120 minutes after the incision or abrasion is made to an area of skin. In some embodiments of each or any of the above- or below-mentioned embodiments, the fresh wound is an incision and the administering is performed along a length of the incision about 1 minute to about 365 after receiving the wound. In some embodiments of each or any of the above- or below-mentioned embodiments, the wound is to be sutured and the administering is performed prior to and/or after suturing of the area of skin that has received the wound.

In a sixth aspect, a method of treating or preventing a keloid in a subject in need thereof is provided, the method comprising administering a composition comprising tropoelastin to an area of skin that has received a wound, wherein the administering is performed about 1 minute to about 365 days after the patient has received the wound, and wherein the subject is prone to keloids.

In some embodiments of each or any of the above- or below-mentioned embodiments, the wound is an abrasion and the composition is administered on an area of skin around the abrasion in parallel to the wound, wherein the administering is performed around a circumference of the wound at about 0.7 cm to about 0.8 cm intervals along the circumference of the wound, and about 0.1 cm, about 0.2 cm, about 0.3 cm, about 0.4 cm, about 0.5 cm, about 0.6 cm, about 0.7 cm, about 0.8 cm about 0.9 cm, about 1 cm, about 1.1 cm, about 1.2 cm, about 1.3 cm, about 1.4 cm or about 1.5 cm away from the wound.

In some embodiments of each or any of the above- or below-mentioned embodiments, the administering is performed about 1 minute, 5 minutes, 10 minutes, 20 minutes, 30 minutes, 60 minutes, 120 minutes after the wound is made. In some embodiments of each or any of the above- or below-mentioned embodiments, the wound is an incision and the administering is performed along a length of the incision about 1 minute to about 365 after receiving the wound. In some embodiments of each or any of the above- or below-mentioned embodiments, the composition is administered as an injection beneath the proposed incision site. In some embodiments of each or any of the above- or below-mentioned embodiments, the composition is injected at about 1 mm, about 2 mm, about 3 mm, about 4 mm, about 5 mm, about 6 mm, about 7 mm, about 8 mm, about 9 mm or about 10 mm, below the wound or proposed incision site.

In a seventh aspect, a method of incising an area of skin on a subject in need thereof is provided. The method comprises making an incision to the area of skin on the subject and administering a composition comprising tropoelastin to the area of skin.

In an eight aspect, a method for preventing scarring in a subject in need thereof is provided. The method comprises administering a composition comprising tropoelastin along a length of an incision site, wherein the subject is a poor wound healer.

In a ninth aspect, a method for preventing scarring in a subject having or receiving an incision is provided. The method comprises identifying the subject as a poor wound healer or a good wound healer and administering a composition comprising tropoelastin along a length of the incision, where the subject is identified as a poor wound healer.

### BRIEF DESCRIPTION OF THE DRAWINGS

The foregoing summary, as well as the following detailed description of the disclosure, will be better understood when read in conjunction with the appended figures. For the purpose of illustrating the disclosure, shown in the figures are embodiments which are presently preferred. It should be understood, however, that the disclosure is not limited to the precise arrangements, examples and instrumentalities shown.

FIG. 1 shows administration of a composition comprising tropoelastin after a patient has received an incision on both sides along the incision length. The injections are performed parallel to the incision. As shown, the composition comprising tropoelastin was injected around both sides of an area of skin that received an incision. The skin was marked up at intervals of 0.7-0.8 cm to indicate positions where the injections of the composition should be administered. Intradermal retrograde injections were then performed parallel to and abutting the skin edge. The dashed line is indicative of the proposed incision site. The small ovals represent the injection sites.

FIG. 2 shows a schematic demonstrating rhTE injections around the area of the proposed incision sites. The dashed line is indicative of the proposed incision site. The small ovals represent the injection sites. As shown, the injection sites are placed at intervals on each side of the proposed incision site as well as the two ends of the incision site.

FIG. 3 shows representative images that illustrate a biopsy wound healing scale with four categories of wound healing. A score of 1 (“Poor”) designates a healed wound comprising a large area, an obvious sharp scar border, irregular scar contour, raised scarring relative to the surrounding skin, and/or mismatached scar color relative to the surrounding skin. A score of 2 (“Average”) designates a wound comprising a discernable scar border, minimally raised scarring, and with some color mismatch present. A score of 3 (“Good”) designates a healed wound comprising a difficult to discern scar border, no discernable raised scarring relative to the surrounding skin, no discernable color mismatch relative to the surrounding skin, and with a texture/wrinkle pattern similar to the adjacent skin.

FIG. 4 shows biopsy wound healing results at day 336 (“D336”) or day 504 (“D504”) following treatment.

### DEFINITIONS

Unless defined otherwise, all technical and scientific terms used herein have the same meaning as commonly understood by one of ordinary skill in the art to which the inventions pertain.

As used herein, “a” or “an” may mean one or more than one. “About” as used herein when referring to a measurable value is meant to encompass variations of +20% or +10%, more preferably +5%, even more preferably +1%, and still more preferably +0.1% from the specified value.

As used herein, except where the context requires otherwise, the term ‘comprise’ and variations of the term, such as “comprising,” “comprises” and “comprised,” are not intended to exclude further additives, components, integers or steps.

As used herein, “scarring,” “fibrosis,” or “fibrotic response” may refer to formation of fibrous (scar) tissue in response to injury or medical intervention.

A “scar,” as used herein, may refer to a mark left on the skin or within body tissue where a wound, burn, or sore has not healed completely and fibrous connective tissue has developed.

The terms, “treat”, “treating”, or “treatment” as used herein refer to at least partially inhibiting a condition including, a clinical symptom of the condition such as a scar or keloid, i.e., arresting or reducing the development of the condition or its clinical symptoms; or relieving the condition, i.e., causing regression of the condition or its clinical symptoms including, a decrease (e.g., a significant decrease including, for example, a statistically significant decrease) in the size, shape, thickness, and/or color of the scar or keloid. Any system used to score a scar is contemplated for use in the methods disclosed herein. For example, a scar may be scored on the Vancouver Scar Scale, Visual Analogue Scale with Scar Ranking, Patient and Observer Scar Assessment Scale, Manchester Scale, or Stony Brook Scar Evaluation Scale (see, e.g., Fearmonti et al., (2011) Plast. Reconstr. Surg. 127(1): 242-247). The treating may improve the score given to a scar (e.g., move the score closer to that of a score given to normal skin) based on any of the aforementioned scales including, as compared to an otherwise identical scar (e.g., a scar from a similar anatomic region of a subject) not treated with the composition disclosed herein.

The terms “prevent”, “prevention”, or “preventing” as used herein refer to a suppression or a reduction, either temporarily or permanently, in the onset of a clinical symptom of a condition such as a scar or keloid (i.e., causing at least one of the clinical symptoms of a condition such as a scar or a keloid not to develop in a subject that may be predisposed to the condition but does not yet experience or display symptoms of the condition). Such preventing need not be absolute to be useful. Prevention of scars or keloids by the compositions disclosed herein includes a decrease (e.g., a significant decrease including, for example, a statistically significant decrease) in the size, shape, thickness, and/or color of the scar or keloid as compared to skin not treated with the compositions. In some embodiments herein, a method is provided to prevent keloid scarring after an incision or a fresh wound. In some embodiments of any one of each or any of the above- or below-mentioned embodiments, the wound is a fresh wound of accidental nature and the composition comprising tropoelastin is administered after the occurrence of the fresh wound, wherein the composition is administered between a time range of about 1 minute to about 10 years after the wound has been made. In some embodiments of any one of each or any of the above- or below-mentioned embodiments, the methods described prevents atrophic scars, scar contractures, hypertrophic scars, and keloids in a subject. In some embodiments of any one of each or any of the above- or below-mentioned embodiments, the subject is prone to developing atrophic scars, scar contractures, hypertrophic scars, and/or keloids. In some embodiments of any one of each or any of the above- or below-mentioned embodiments, the subject has previously developed atrophic scars, scar contractures, hypertrophic scars, and/or keloids in past scarring.

The term “pre-treatment” refers to an act or instance of treating someone or something in advance for a preliminary or preparatory treatment. In the embodiments herein, a pre-treatment method is provided for reducing or preventing scarring and scar discoloration.

The term “post-treatment” refers to an act or instance of treating someone soon after a treatment or an accident. In the embodiments herein, a post-treatment method is provided for reducing or preventing scarring or scar discoloration. This may be used after a surgical procedure or on a fresh wound. Without being limiting, a fresh wound may be an abrasion, cut or an incision, for example. In some embodiments of any one of each or any of the above- or below-mentioned embodiments, the incision made for a surgical procedure.

The term “tropoelastin” refers to a protein from which elastin is formed. Tropoelastin may be monomeric. Tropoelastin is generally not cross-linked, covalently or otherwise. Tropoelastin may reversibly coacervate. Thus, tropoelastin is distinguished from elastin because elastin consists of covalently cross linked tropoelastin which cannot reversibly coacervate. The tropoelastin may be human tropoelastin. Tropoelastin may be synthetic, for example it may be derived from recombinant expression or other synthesis, or it may be obtained from a natural source such as porcine aorta. As generally known in the art, tropoelastin may exist in the form of a variety of fragments. In some embodiments of any one of each or any of the above- or below-mentioned embodiments, the tropoelastin comprises the sequence set forth in any one of SEQ ID NOs: 1-15.

The term “hyaluronic acid” or “HA” may include hyaluronic acid and any of its hyaluronate salts, including, for example, sodium hyaluronate (the sodium salt), potassium hyaluronate, magnesium hyaluronate, and calcium hyaluronate. Hyaluronic acid from a variety of sources may be used herein. For example, hyaluronic acid may be extracted from animal tissues, harvested as a product of bacterial fermentation, or produced in commercial quantities by bioprocess technology. In some embodiments of any one of each or any of the above- or below-mentioned embodiments, the tropoelastin comprises the sequence set forth in SEQ ID NO: 1.

The term “Fitzpatrick scale” is given its plain and ordinary meaning, in view of this paper and without limitation, may refer to a scientific skin type classification. The Fitzpatrick scale has several different types from types 1-6. This scale is used by dermatologists and aesthetic medicine practitioners to determine which treatments are best suited for different skin types. The scale was developed to measure how skin reacts to ultraviolet light such as sun exposure. Additionally, this knowledge can be used by aesthetic doctors and laser technicians to know before administering a laser or another type of treatment. Type 1—typically has light, ivory skin but when exposed to the sun always burns and peels but never tans. Type 2—has a light, fair complexion and burns quickly when exposed to the sun and rarely tans. Type 3—usually has a beige tint to the skin and may burn when exposed to the sun but is capable of tanning. Type 4—has an olive skin or light brown tone and will not freckle when exposed to the sun. This person rarely gets a sun burn and tans regularly. Type 5—has a dark brown or black skin tone, rarely gets a sun burn and always tans under sun exposure. Type 6—has black and is the darkest skin tone. This person never burns and tans quickly when exposed to the sun. This helpful scale may be used by dermatologists to gauge certain skin's reaction to the sun and how to more quickly identify potentially malignant sunspots. Aesthetic doctors may also use the scale to determine the effectiveness of treatments on different skin types. In some embodiments of any one of each or any of the above- or below-mentioned embodiments, the methods described herein are pre-operative treatment to prevent scarring of surgical wounds prior to the incision and may be used to prevent or treat scarring caused by the surgery. In some embodiments of any one of each or any of the above- or below-mentioned embodiments, the pre-treatment methods in which compositions are administered to a patient in need prior to a surgical procedure to prevent excessive scarring. In some embodiments of any one of each or any of the above- or below-mentioned embodiments, the compositions are administered to a patient in need after the patient has received a fresh wound such as an abrasion, incision or a cut. Without being limiting, a fresh wound may be an abrasion, cut or an incision, for example. The patient in need may have the skin type classification of any one of types 1-6 on the Fitzpatrick scale.

The term “keloid” is given its plain and ordinary meaning, in view of this paper and without limitation, may refer to a raised type of a scar. When skin is injured, fibrous tissue called scar tissue may grow excessively and are known to grow much larger than a wound that caused the scar. For those who are prone to keloids, the keloid may be caused by a cut, burn acne, for example. In rare cases keloids can also appear without injury and are known as “spontaneous keloids.” Although keloids are not harmful to one's health, they may lead to itchiness, discomfort and tenderness. In some cases, the keloids are hardened, tight scar tissue that may impede movement. In some embodiments of any one of each or any of the above- or below-mentioned embodiments, the methods comprise administering a composition comprising tropoelastin at a localized area around a keloid that will be surgically removed (e.g., prior to removal of the keloid). In some embodiments of any one of each or any of the above- or below-mentioned embodiments, pre-treatment with the compositions disclosed herein prevents keloid re-occurrence in the patient receiving the pre-treatment. In some embodiments of any one of each or any of the above- or below-mentioned embodiments, the composition is administered as an injection beneath proposed keloid site, wherein the composition is injected at about 1 mm, about 2 mm, about 3 mm, about 4 mm, about 5 mm, about 6 mm, about 7 mm, about 8 mm, about 9 mm, or about 10 mm below the keloid site.

Patients with darker pigmentation may have a higher prevalence in the formation of keloids (Halim et. al., Archives of Plastic Surgery, Vol. 39, No. 3, pp. 184-189; incorporated by reference in its entirety herein). For example, it is the fifth most common skin disease in adult black patients in the United Kingdom and the most common skin disease among ethnic Chinese patients in Asia. (Halim et al.) In some embodiments of any one of each or any of the above- or below-mentioned embodiments, the method prevents keloids in a subject, wherein the subject is prone to forming keloids and wherein the subject comprises skin on the Fitzpatrick scale of 3-6. In some embodiments of any one of each or any of the above- or below-mentioned embodiments, the subject is of African or Asian descent. In some embodiments of any one of each or any of the above- or below-mentioned embodiments, the subject has previously developed keloid scars and the method prevents re-occurrence of keloid scarring.

Pathological scars may be described as scars as those that have “an evolution in time that is completely different from the regular course of wound healing” including but not limited to widespread scars, atrophic scars, scar contractures, hypertrophic scars, and keloids (Fearmonti et al.; Pathologic and Nonpathologic Scarring, 127(1) pp. 242-247; incorporated by reference in its entirety herein). In some embodiments of the above- or below-mentioned embodiments, the treatment for preventing scarring is for a patient that has a history for developing atrophic scars, scar contractures, hypertrophic scars, and/or keloids.

The term “intradermal implant” is an implant that may go underneath the thin layer of the skin. Without being limiting, this may be in the area of skin such as the dermis, subdermis, hypodermis or subcutaneous layer, for example.

As described herein, “localized delivery” refers to the administration of the composition into or near the tissue in need of the therapeutic composition. This site may be an area that is to receive a surgical incision. It may also be a site within proximity of a keloid, in which an individual will receive a surgical treatment or a laser to remove a keloid.

### DETAILED DESCRIPTION

The inventors have developed methods to treat and/or prevent scar formation and/or scar discoloration resulting from an incision made to an area of skin. The methods may comprise steps for a pre-treatment as well as a post-treatment to prevent scar formation and/or scar discoloration. The methods may be performed prior to a surgical procedure that introduces an incision to an area of skin. Further provided herein are compositions for preventing and reducing scarring (e.g., excessive scarring). It is also contemplated that the compositions prevent scar discoloration.

The area of skin to receive the injection may be the exact site that is to receive the incision. For example, prior to surgery, a surgeon will carry out marking of a patient's skin in order to clearly identify the intended site of the incision, the proposed incision site, or as near as possible to the intended site. As described in the embodiments herein, the site for injection of a composition comprising tropoelastin may be directly into the site of the proposed incision site, at, and/or around the proposed incision site at both sides of the proposed incision site, and at the ends of the incision site, and so that the increments of injection run parallel to the proposed incision site at both sides.

The injections may be administered directly into the site of the proposed incision site, or at least about 0.1 cm, about 0.5 cm, about 1 cm or about 1.5 cm away from the site to receive the incision (e.g. every 0.1 cm, 0.2 cm, 0.3 cm, 0.4 cm, 0.5 cm, 0.6 cm, 0.7 cm, 0.8 cm, 0.9 cm or 1.0 cm along the length of the incision site on one or both sides of the incision and or on the ends of the incision site). In some embodiments, the composition is administered as an injection beneath, or in an area about the site to receive the incision, the composition is injected at about 1 mm, about 2 mm, about 3 mm, about 4 mm, about 5 mm, about 6 mm, about 7 mm, about 8 mm, about 9 mm or about 10 mm below the site to receive the incision. In other embodiments, the injection is in the area of a keloid, it is injected about 1 mm, about 2 mm, about 3 mm, about 4 mm, about 5 mm, about 6 mm, about 7 mm, about 8 mm, about 9 mm or about 10 mm below the keloid. In some embodiments, the composition may also be injected about 0.1 cm, about 0.2 cm, about 0.3 cm, about 0.4 cm, about 0.5 cm, about 0.6 cm, about 0.7 cm, about 0.8 cm about 0.9 cm, about 1 cm, about 1.1 cm, about 1.2 cm, about 1.3 cm, about 1.4 cm or about 1.5 cm away from the ends of the proposed incision site. In some embodiments, the composition is injected about 0.1 cm away from the site to receive the incision.

The composition may comprise 30 mg/ml recombinant human tropoelastin crosslinked with 0.5% hyaluronic acid and may be administered in a volume of about 1 μL to about 100 μL. In some embodiments, the area of skin receives 1 μL to about 100 μL of the composition per square centimeter of skin.

In some embodiments, the composition is injected about 0.5 cm away from the site to receive the incision, wherein the composition comprises 30 mg/ml recombinant human tropoelastin crosslinked with 0.5% hyaluronic acid, and wherein the composition is administered in a volume of about 25 μL to about 50 μL in increments along the length of the proposed incision site.

In some embodiments, the composition is injected about 0.5 cm away from the site to receive the incision, wherein the composition comprises 30 mg/ml recombinant human tropoelastin crosslinked with 0.5% derivatized hyaluronic acid, and wherein the composition is administered in a volume of about 25 μL to about 50 μL in increments along the length of the proposed incision site.

In some embodiments, the composition is injected about 1.0 cm away from the site to receive the incision, wherein the composition comprises 30 mg/ml recombinant human tropoelastin crosslinked with 0.5% derivatized hyaluronic acid, and wherein the composition is administered in a volume of about 50 μL to about 75 μL in increments along the length of the proposed incision site and/or the ends of the proposed incision site.

In some embodiments, the composition is injected about 1.5 cm away from the site to receive the incision, wherein the composition comprises 30 mg/ml recombinant human tropoelastin crosslinked with 0.5% derivatized hyaluronic acid, and wherein the composition is administered in a volume of about 75 μL to about 100 μL in increments along the length of the proposed incision site.

In some embodiments, the composition is injected about 1.0 cm away from the site to receive the incision, wherein the composition comprises 30 mg/ml recombinant human tropoelastin crosslinked with 0.5% hyaluronic acid, and wherein the composition is administered in a volume of about 50 μL to about 75 μL in increments along the length of the proposed incision site.

In some embodiments, the composition is injected about 1.5 cm away from the site to receive the incision, wherein the composition comprises 30 mg/ml recombinant human tropoelastin crosslinked with 0.5% hyaluronic acid, and wherein the composition is administered in a volume of about 75 μL to about 100 μL in increments along the length of the proposed incision site.

The volume of composition injected is about 1 μl to 100 μl of composition per square centimeter along the incision length.

The injections may be directly into the site that is to receive the incision. In some embodiments, the composition comprising tropoelastin is administered by injections to every 0.1 cm to 2.0 cm intervals along the proposed incision site, so that they are parallel to the incision site in about 0.1 to about 2.0 cm increments along the site of injection, wherein the injections occur at about 0.1 cm, about 0.5 cm, about 1.0 cm about 1.5 cm or about 2.0 cm away from the proposed incision site. In some embodiments, the volume of composition injected is about 1 μl to 100 μl of composition per square centimeter along the incision or wound length.

The embodiments described herein are directed to prevention of a scar in an area of skin that will receive an incision, such as a surgical incision or in a fresh wound, such as a scrape or a cut that occurred accidentally, so that no scar (or a scar of reduced size, thickness, or color) is formed when the incision heals. It is also contemplated that embodiments described also prevent scar formation as the result of surgery. It is known that healing of wounds may require extra-cellular biosynthesis and the production of fibrous connective tissue, leading to scarring.

Additionally, the compositions may also prevent scarring that may be a result due to the surgery, such as the build-up of scar tissue that occurs from the surgery. The compositions disclosed herein when administered to an area of skin set to receive an incision surprisingly prevent and/or reduce the formation of scarring at the area of skin after the incision is made and subsequently heals. In some embodiments, the composition is administered above the incision site, beneath the proposed incision site and/or the area of skin around the proposed incision site, along the length of the proposed incision site.

The composition may be applied as an intradermal implant. The composition may be administered as a dermal implant, subdermal implant, within the hypodermis and at the subcutaneous level, for example. The composition may be administered within the layers of the epidermis, which include the stratum basale, stratum spinosum, stratum granulosum, stratum lucidum and the stratum corneum. The composition may be administered within the layers of the dermis. The dermis is the middle layer of the three layers of the skin between the epidermis and the subcutaneous tissues. The thickness of the dermis ranges from about 0.6 millimeters thick (i.e. eyelids) to about 3 millimeters thick (i.e. soles of the feet). The composition may be administered within the subcutaneous tissues, which include the deepest innermost layer of skin. The thickness of this layer varies on its location on the body. Thus, the composition may be administered in all described areas of the skin.

The compositions described herein may also be used in the treatment of fresh wounds such as scrapes and cuts. In some embodiments, a post-treatment is provided, wherein the composition is administered as an intradermal implant, a dermal implant, subdermal implant, within the hypodermis and/or at the subcutaneous level. The composition may be administered to the area of skin after a wound is made to the area of skin. In some embodiments, the wound is an abrasion, scrape or a cut. In some embodiments, the cut has a depth of about 0.1 to about 3 cm in depth. In some embodiments, the composition is administered as an injection beneath or around the area for incision. In some embodiments, the composition is administered by injection at about 0.1 to about 2.0 cm intervals (e.g., about 0.7 to about 0.8 cm intervals), along the area of skin that has received the wound, or along each side of the area of skin that has received a wound. In some embodiments, the composition is placed as an intradermal implant, a dermal implant, subdermal implant, within the hypodermis and/or at the subcutaneous level, within 0.1 cm, 0.2 cm, 0.3 cm, 0.4 cm, 0.5 cm, 0.6 cm, 0.7 cm, 0.8 cm, 0.9 cm or 1.0 cm from the wound at any one of the described layers of skin. In some embodiments, the cut has a depth of greater than 3 cm. In some embodiments, the composition is administered above the wound, beneath the wound or along the length of the proposed incision site.

It is also contemplated that administration of the composition comprising tropoelastin further prevents scarring that may be a result from surgery. In some embodiments, the methods herein further prevent the formation of surgical scar tissue.

In some embodiments, the composition is administered after a surgical incision is made, either prior to suturing or after suturing of the surgical incision. In some embodiments, the composition is re-administered until the skin is healed and there is no scarring.

In some embodiments, the methods utilize the SHELδ26A tropoelastin analogue (WO 1999/03886) for the various applications described herein including for the compositions that are used in the described methods. The amino acid sequence of SHELδ26A is:

(SEQ ID NO: 1)
GGVPGAIPGGVPGGVFYPGAGLGALGGGALGPGGKPLKPVPGGLAGAGLG
AGLGAFPAVTFPGALVPGGVADAAAAYKAAKAGAGLGGVPGVGGLGVSAG
AVVPQPGAGVKPGKVPGVGLPGVYPGGVLPGARFPGVGVLPGVPTGAGVK
PKAPGVGGAFAGIPGVGPFGGPQPGVPLGYPIKAPKLPGGYGLPYTTGKL
PYGYGPGGVAGAAGKAGYPTGTGVGPQAAAAAAAKAAAKFGAGAAGVLPG
VGGAGVPGVPGAIPGIGGIAGVGTPAAAAAAAAAAKAAKYGAAAGLVPGG
PGFGPGVVGVPGAGVPGVGVPGAGIPVVPGAGIPGAAVPGVVSPEAAAKA
AAKAAKYGARPGVGVGGIPTYGVGAGGFPGFGVGVGGIPGVAGVPSVGGV
PGVGGVPGVGISPEAQAAAAAKAAKYGVGTPAAAAAKAAAKAAQFGLVPG
VGVAPGVGVAPGVGVAPGVGLAPGVGVAPGVGVAPGVGVAPGIGPGGVAA
AAKSAAKVAAKAQLRAAAGLGAGIPGLGVGVGVPGLGVGAGVPGLGVGAG
VPGFGAVPGALAAAKAAKYGAAVPGVLGGLGALGGVGIPGGVVGAGPAAA
AAAAKAAAKAAQFGLVGAAGLGGLGVGGLGVPGVGGLGGIPPAAAAKAAK
YGAAGLGGVLGGAGQFPLGGVAARPGFGLSPIFPGGACLGKACGRKRK.

In some embodiments, the tropoelastin isoform is the SHEL isoform (WO 1994/14958; included by reference in its entirety herein); SMGGVPGAIPGGVPGGVFYPGAGLGALGGGALGPGGKPLKPVPGGLAGAGLGAGLGA FPAVTFPGALVPGGVADAAAAYKAAKAGAGLGGVPGVGGLGVSAGAVVPQPGAGVK PGKVPGVGLPGVYPGGVLPGARFPGVGVLPGVPTGAGVKPKAPGVGGAFAGIPGVGPF GGPQPGVPLGYPIKAPKLPGGYGLPYTTGKLPYGYGPGGVAGAAGKAGYPTGTGVGPQ AAAAAAAKAAAKFGAGAAGVLPGVGGAGVPGVPGAIPGIGGIAGVGTPAAAAAAAAA AKAAKYGAAAGLVPGGPGFGPGVVGVPGAGVPGVGVPGAGIPVVPGAGIPGAAVPGV VSPEAAAKAAAKAAKYGARPGVGVGGIPTYGVGAGGFPGFGVGVGGIPGVAGVPSVG GVPGVGGVPGVGISPEAQAAAAAKAAKYGVGTPAAAAAKAAAKAAQFGLVPGVGVA PGVGVAPGVGVAPGVGLAPGVGVAPGVGVAPGVGVAPGIGPGGVAAAAKSAAKVAA KAQLRAAAGLGAGIPGLGVGVGVPGLGVGAGVPGLGVGAGVPGFGAGADEGVRRSLS PELREGDPSSSQHLPSTPSSPRVPGALAAAKAAKYGAAVPGVLGGLGALGGVGIPGGVV GAGPAAAAAAAKAAAKAAQFGLVGAAGLGGLGVGGLGVPGVGGLGGIPPAAAAKAA KYGAAGLGGVLGGAGQFPLGGVAARPGFGLSPIFPGGACLGKACGRKRK (SEQ ID NO: 2) or a protease resistant derivative of the SHEL or SHELδ26A isoforms (WO 2000/04043; included by reference in its entirety herein). As described in WO 2000/04043, the protein sequences of tropoelastin described may have a mutated sequence that leads to a reduced or eliminated susceptibility to digestion by proteolysis. Without being limiting, the tropoelastin amino acid sequence has a reduced or eliminated susceptibility to serine proteases, thrombin, kallikrein, metalloproteases, gelatinase A, gelatinase B, serum proteins, trypsin or elastase, for example. In some embodiments of any one of each or any of the above- or below-mentioned embodiments, the tropoelastin comprises a sequence set forth in SEQ ID NO: 3 (SHELδ26A isoform):
GGVPGAIPGGVPGGVFYPGAGLGALGGGALGPGGKPLKPVPGGLAGAGLGAGL GAFPAVTFPGALVPGGVADAAAAYKAAKAGAGLGGVPGVGGLGVSAGAVVPQPGAG VKPGKVPGVGLPGVYPGGVLPGARFPGVGVLPGVPTGAGVKPKAPGVGGAFAGIPGVG PFGGPQPGVPLGYPIKAPKLPGGYGLPYTTGKLPYGYGPGGVAGAAGKAGYPTGTGVG PQAAAAAAAKAAAKFGAGAAGVLPGVGGAGVPGVPGAIPGIGGIAGVGTPAAAAAAA AAAKAAKYGAAAGLVPGGPGFGPGVVGVPGAGVPGVGVPGAGIPVVPGAGIPGAAVP GVVSPEAAAKAAAKAAKYGARPGVGVGGIPTYGVGAGGFPGFGVGVGGIPGVAGVPS VGGVPGVGGVPGVGISPEAQAAAAAKAAKYGVGTPAAAAAKAAAKAAQFGLVPGVG VAPGVGVAPGVGVAPGVGLAPGVGVAPGVGVAPGVGVAPGIGPGGVAAAAKSAAKV AAKAQLRAAAGLGAGIPGLGVGVGVPGLGVGAGVPGLGVGAGVPGFGAVPGALAAA KAAKYGAAVPGVLGGLGALGGVGIPGGVVGAGPAAAAAAAKAAAKAAQFGLVGAAG LGGLGVGGLGVPGVGGLGGIPPAAAAKAAKYGAAGLGGVLGGAGQFPLGGVAARPGF GLSPIFPGGACLGKACGRKRK (SEQ ID NO: 3). In some embodiments, the tropoelastin comprises a sequence set forth in SEQ ID NO: 4 (SHELδ mod isoform):

(SEQ ID NO: 4)
GGVPGAVPGGVPGGVFYPGAGFGAVPGGVADAAAAYKAAKAGAGLGGVPG
VGGLGVSAGAVVPQPGAGVKPGKVPGVGLPGVYPGFGAVPGARFPGVGVL
PGVPTGAGVKPKAPGVGGAFAGIPGVGPFGGPQPGVPLGYPIKAPKLPGG
YGLPYTTGKLPYGYGPGGVAGAAGKAGYPTGTGVGPQAAAAAAAKAAAKF
GAGAAGFGAVPGVGGAGVPGVPGAIPGIGGIAGVGTPAAAAAAAAAAKAA
KYGAAAGLVPGGPGFGPGVVGVPGFGAVPGVGVPGAGIPVVPGAGIPGAA
GFGAVSPEAAAKAAAKAAKYGARPGVGVGGIPTYGVGAGGFPGFGVGVGG
IPGVAGVPSVGGVPGVGGVPGVGISPEAQAAAAAKAAKYGVGTPAAAAAK
AAAKAAQFGLVPGVGVAPGVGVAPGVGVAPGVGLAPGVGVAPGVGVAPGV
GVAPGIGPGGVAAAAKSAAKVAAKAQLRAAAGLGAGIPGLGVGVGVPGLG
VGAGVPGLGVGAGVPGFGAVPGALAAAKAAKYGAVPGVLGGLGALGGVGI
PGGVVGAGPAAAAAAAKAAAKAAQFGLVGAAGLGGLGVGGLGVPGVGGLG
GIPPAAAAKAAKYGAAGLGGVLGGAGQFPLGGVAARPGFGLSPIFPGGAC
LGKACGRKRK.

Tropoelastin analogues generally have a sequence that is homologous to a human tropoelastin sequence. Percentage identity between a pair of sequences may be calculated by the algorithm implemented in the BESTFIT computer program. Another algorithm that calculates sequence divergence has been adapted for rapid database searching and implemented in the BLAST computer program. In comparison to the human sequence, the tropoelastin polypeptide sequence may be about 60% identical at the amino acid level, 70% or more identical at the amino acid level, 80% or more identical at the amino acid level, 90% or more identical at the amino acid level, 95% or more identical at the amino acid level, 97% or more identical at the amino acid level, or greater than 99% identical at the amino acid level.

Conservative amino acid substitutions (e.g., Glu/Asp, Val/Ile, Ser/Thr, Arg/Lys, Gln/Asn) may also be considered when making comparisons because the chemical similarity of these pairs of amino acid residues are expected to result in functional equivalency in many cases. Amino acid substitutions that are expected to conserve the biological function of the polypeptide would conserve chemical attributes of the substituted amino acid residues such as hydrophobicity, hydrophilicity, side-chain charge, or size.

Recombinant forms of tropoelastin can be produced as shown in WO 1999/03886. These sequences are:

(SEQ ID NO: 5)
SMGGVPGAIPGGVPGGVFYPGAGLGALGGGALGPGGKPLKPVPGGLAGAG
LGAGLGAFPAVTFPGALVPGGVADAAAAYKAAKAGAGLGGVPGVGGLGVS
AGAVVPQPGAGVKPGKVPGVGLPGVYPGGVLPGARFPGVGVLPGVPTGAG
VKPKAPGVGGAFAGIPGVGPFGGPQPGVPLGYPIKAPKLPGGYGLPYTTG
KLPYGYGPGGVAGAAGKAGYPTGTGVGPQAAAAAAAKAAAKFGAGAAGVL
PGVGGAGVPGVPGAIPGIGGIAGVGTPAAAAAAAAAAKAAKYGAAAGLVP
GGPGFGPGVVGVPGAGVPGVGVPGAGIPVVPGAGIPGAAVPGVVSPEAAA
KAAAKAAKYGARPGVGVGGIPTYGVGAGGFPGFGVGVGGIPGVAGVPSVG
GVPGVGGVPGVGISPEAQAAAAAKAAKYGVGTPAAAAAKAAAKAAQFGLV
PGVGVAPGVGVAPGVGVAPGVGLAPGVGVAPGVGVAPGVGVAPGIGPGGV
AAAAKSAAKVAAKAQLRAAAGLGAGIPGLGVGVGVPGLGVGAGVPGLGVG
AGVPGFGAGADEGVRRSLSPELREGDPSSSQHLPSTPSSPRVPGALAAAK
AAKYGAAVPGVLGGLGALGVGIPGGVVGAGPAAAAAAAKAAAKAAQFGLV
GAAGLGGLGVGGLGVPGVGGLGGIPPAAAAKAAKYGAAGLGGVLGGAGQF
PLGGVAARPGFGLSPIFPGGACLGKACGRKRK;
(SEQ ID NO: 6)
GGVPGAIPGGVPGGVFYPGAGLGALGGGALGPGGKPLKPVPGGLAGAGLG
AGLGAFPAVTFPGALVPGGVADAAAAYKAAKAGAGLGGVPGVGGLGVSAG
AVVPQPGAGVKPGKVPGVGLPGVYPGGVLPGARFPGVGVLPGVPTGAGVK
PKAPGVGGAFAGIPGVGPFGGPQPGVPLGYPIKAPKLPGGYGLPYTTGKL
PYGYGPGGVAGAAGKAGYPTGTGVGPQAAAAAAAKAAAKFGAGAAGVLPG
VGGAGVPGVPGAIPGIGGIAGVGTPAAAAAAAAAAKAAKYGAAAGLVPGG
PGFGPGVVGVPGAGVPGVGVPGAGIPVVPGAGIPGAAVPGVVSPEAAAKA
AAKAAKYGARPGVGVGGIPTYGVGAGGFPGFGVGVGGIPGVAGVPSVGGV
PGVGGVPGVGISPEAQAAAAAKAAKYGVGTPAAAAAKAAAKAAQFGLVPG
VGVAPGVGVAPGVGVAPGVGLAPGVGVAPGVGVAPGVGVAPGIGPGGVAA
AAKSAAKVAAKAQLRAAAGLGAGIPGLGVGVGVPGLGVGAGVPGLGVGAG
VPGFGAVPGALAAAKAAKYGAAVPGVLGGLGALGGVGIPGGVVGAGPAAA
AAAAKAAAKAAQFGLVGAAGLGGLGVGGLGVPGVGGLGGIPPAAAAKAAK
YGAAGLGGVLGGAGQFPLGGVAARPGFGLSPIFPGGACLGKACGRKRK;
(SEQ ID NO: 7)
MGGVPGAVPGGVPGGVFYPGAGFGAVPGGVADAAAAYKAAKAGAGLGGVP
GVGGLGVSAGAVVPQPGAGVKPGKVPGVGLPGVYPGFGAVPGARFPGVGV
LPGVPTGAGVKPKAPGVGGAFAGIPGVGPFGGPQPGVPLGYPIKAPKLPG
GYGLPYTTGKLPYGYGPGGVAAAGKAGYPTGTGVGPQAAAAAAAKAAAKF
GAGAAGFGAVPGVGGAGVPGVPGAIPGIGGIAGVGTPAAAAAAAAAAKAA
KYGAAAGLVPGGPGFGPGVVGVPGFGAVPGVGVPGAGIPVVPGAGIPGAA
GFGAVSPEAAAKAAAKAAKYGARPGVGVGGIPTYGVGAGFFPGFGVGVGG
IPGVAGVPSVGGVPGVGGVPGVGISPEAQAAAAAKAAKYGVGTPAAAAAK
AAAKAAQFGLVPGVGVAPGVGVAPGVGVAPGVGLAPGVGVAPGVGVAPGV
GVAPGIGPGGVAAAAKSAAKVAAKAQLRAAAGLGAGIPGLGVGVGVPGLG
VGAGVPGLGVGAGVPGFGAVPGALAAAKAAKYGAVPGVLGGLGALGGVGI
PGGVVGAGPAAAAAAAKAAAKAAQFGLVGAAGLGGLGVGGLGVPGVGGLG
GIPPAAAAKAAKYGAAGLGGVLGGAGQFPLGGVAARPGFGLSPIFPGGAC
LGKACGRKRK;
(SEQ ID NO: 8)
SAMGGVPGALAAAKAAKYGAAVPGVLGGLGALGGVGIPGGVVGAGPAAAA
AAAKAAAKAAQFGLVGAAGLGGLGVGGLGVPGVGGLGGIPPAAAAKAAKY
GAAGLGGVLGGAGQFPLGGVAARPGFGLSPIFPGGACLGKACGRKRK;
(SEQ ID NO: 9)
SAMGALVGLGVPGLGVGAGVPGFGAGADEGVRRSLSPELREGDPSSSQH
LPSTPSSPRVPGALAAAKAAKYGAAVPGVLGGLGALGGVGIPGGVVGAG
PAAAAAAAKAAAKAAQFGLVGAAGLGGLGVGGLGVPGVGGLGGIPPAAA
AKAAKYGAAGLGGVLGGAGQFPLGGVAARPGFGLSPIFPGGACLGKACG
RKRK;
(SEQ ID NO: 10)
GIPPAAAAKAAKYGAAGLGGVLGGAGQFPLGGVAARPGFGLSPIFPGGAC
LGKACGRKRK;
(SEQ ID NO: 11)
GAAGLGGVLGGAGQFPLGGVAARPGFGLSPIFPGGACLGKACGRKRK;
(SEQ ID NO: 12)
GADEGVRRSLSPELREGDPSSSQHLPSTPSSPRV;
(SEQ ID NO: 13)
GADEGVRRSLSPELREGDPSSSQHLPSTPSSPRF;
(SEQ ID NO: 14)
AAAGLGAGIPGLGVGVGVPGLGVGAGVPGLGVGAGVPGFGAGADEGVRRS
LSPELREGDPSSSQHLPSTPSSPRVPGALAAAKAAKYGAAVPGVLGGLGA
LGGVGIPGGVVGAGPAAAAAAAKAAAKAAQFGLVGAAGLGGLGVGGLGVP
GVGGLGGIPPAAAAKAAKYGAAGLGGVLGGAGQFPLGGVAARPGFGLSPI
FPGGACLGKACGRKRK;
and
(SEQ ID NO: 15)
AAAGLGAGIPGLGVGVGVPGLGVGAGVPGLGVGAGVPGFGAVPGALAAAK
AAKYGAAVPGVLGGLGALGGVGIPGGVVGAGPAAAAAAAKAAAKAAQFGL
VGAAGLGGLGVGGLGVPGVGGLGGIPPAAAAKAAKYGAAGLGGVLGGAGQ
FPLGGVAARPGFGLSPIFPGGACLGKACGRKRK.

Tropoelastin may be utilized in a form in which it is linked (e.g., covalently linked) to another molecule such as a biopolymer such as hyaluronic acid. In some embodiments, the tropoelastin is cross-linked to hyaluronic acid. In some embodiments, the tropoelastin comprises the amino acid sequence set forth in any one of SEQ ID NOs: 1-15. In some embodiments, the tropoelastin consists of the amino acid sequence set forth in any one of SEQ ID NOs: 1-15.

It is particularly preferred that where tropoelastin is linked to another molecule, the linkage does not impede or limit the biological properties of an unlinked form of tropoelastin.

The purpose of linking tropoelastin with another molecule is typically to enable tropoelastin to be localized to a region and to minimize the likelihood of the tropoelastin diffusing or otherwise migrating from that region.

In some embodiments, the tropoelastin has a specified degree of purity with respect to the amount of tropoelastin in the composition, as compared with amounts of other proteins or molecules in the composition. In some embodiments, the tropoelastin is in a composition that has at least 75% purity, preferably 85% purity, more preferably more than 90% or 95% purity. Fragments of tropoelastin, i.e., truncated forms of a tropoelastin isoform that arise unintentionally through tropoelastin manufacture may be regarded as an impurity in this context.

It will further be understood that in certain embodiments the tropoelastin may be provided in the form of a composition that consists of or consists essentially of tropoelastin, preferably a full-length isoform of tropoelastin. In alternative embodiments, the tropoelastin will be at least about 65% of the length of the relevant tropoelastin isoform, more than about 80% of the full length, more than about 90% or more than about 95% of the full length.

The composition may comprise between 1-100 mg/ml tropoelastin crosslinked with hyaluronic acid (HA). In some embodiments, the composition comprises about 0.5% to about 10% hyaluronic acid. In some embodiments, the composition comprises about 0.2%, about 0.3%, about 0.4%, about 0.5%, about 0.6%, about 0.7%, about 0.8%, 0.9% or about 1% hyaluronic acid (HA). In some embodiments, the tropoelastin is in a monomeric form and is crosslinked with the hyaluronic acid. In some embodiments, the composition comprises about 20 mg/ml, about 30 mg/ml, about 40 mg/ml or about 50 mg/ml recombinant human tropoelastin. In some embodiments, the composition comprises 30 mg/ml recombinant human tropoelastin crosslinked with 0.5% hyaluronic acid. The hyaluronic acid is not limited to derivatized hyaluronic acid. In some embodiments, the composition comprises derivatized HA (dHA) or underivatized HA, to control the extent to which the HA crosslinks with itself and/or the monomeric protein. In some embodiments, the tropoelastin in the composition is cross-linked to hyaluronic acid, wherein the composition comprises derivatised HA or underivatized HA. In some embodiments, the monomeric tropoelastin is released from the composition.

In some embodiments, the HA may comprise, at least one linkable moiety, such as at least one cross-linkable moiety, for example, a carboxyl group, a hydroxyl group, an amine, a thiol, an alcohol, an alkene, an alkyne, a cyano group, or an azide, and/or modifications, derivatives, or combinations thereof.

In some embodiments, the HA may comprise, a spacer group, such that the spacer group can link to the same and/or a second molecule, for example, a second biomolecule or biopolymer.

Hyaluronic acid comprises a polymer of disaccharides composed of D-glucuronic acid and N-acetyl-D-glucosamine linked via alternating β-(1→4) and β-(1→3) glycosidic bonds. The HA may be in the range of about 50 to about 4000 disaccharide units or residues, for example about 2000 to 2500 disaccharide units or residues. In some embodiments of any one of each or any of the above- or below-mentioned embodiments, hyaluronic acid may be used in the range of 200 to 10,000 disaccharide units or residues. In some embodiments of any one of each or any of the above- or below-mentioned embodiments, there may be 10,000 or more repeating disaccharide units in hyaluronic acid.

The molecular weight of the HA may be in the range from about 5 kDa to about 20,000 kDa. In some embodiments of any one of each or any of the above- or below-mentioned embodiments, the HA may be about 5 kDa to about 3000 kDa. In some embodiments herein, the HA is about 8000-1000 kDa. In some embodiments, the HA is about 5 kDa, about 10 kDa, about 20 kDa, about 30 kDa, about 40 kDA, about 50 kDa, about 60 kDa, about 70 kDa, about 80 kDa, about 90 kDa, about 100 kDa, about 200 kDa, about 300 kDa, about 400 kDa, about 500 kDa, about 600 kDa, about 700 kDa, about 800 kDa, about 900 kDa, about 1000 kDa, about 2000 kDa, about 3000 kDa, about 4000 kDa, about 5000 kDa, about 6000 kDa, about 7000 kDa, about 8000 kDa, about 9000 kDa or about 10,000 kDa.

In some embodiments, the HA may be low or high molecular weight HA.

In some embodiments, the composition comprises a combination of low molecular weight HA and high molecular weight HA.

In some embodiments, the HA may be activated and/or modified with an activating agent, such as EDC or allylglycidyl ether, and/or modifying agent, such as NHS, HOBt or Bromine.

In some embodiments, a method of pre-treating skin of a patient to prevent scarring is provided. The method comprises administering a composition comprising tropoelastin to an area of skin that is to receive an incision. The composition is administered in a localized area for localized delivery. In some embodiments, the composition comprises derivatised HA or underivatized HA, to control the extent to which the HA crosslinks with itself and/or the monomeric protein. In some embodiments, the administration of the composition comprises injecting the composition into the area of skin prior to incision. This may be placed as an intradermal implant, a dermal implant, subdermal implant, within the hypodermis and/or at the subcutaneous level, within 0.1 cm, 0.2 cm, 0.3 cm, 0.4 cm, 0.5 cm, 0.6 cm, 0.7 cm, 0.8 cm, 0.9 cm or 1.0 cm from the incision at any one of the described layers of skin. In some embodiments, the composition is administered by injection at about 0.7 to about 0.8 cm intervals, along the area of skin that is to receive the incision, along each side of the incision. In some embodiments, the composition is administered prior to surgery. In some embodiments, the composition is administered to the area of skin between about 1 minute and about 90 days prior to surgery where the incision is made to the area of skin. In some embodiments, the composition is administered to the area of skin between about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, about 31 days, about 32 days, about 33 days, about 34 days, about 35 days, about 36 days, about 37 days, about 38 days, about 39 days, about 40 days, about 41 days, about 42 days, about 43 days, about 44 days, about 45 days, about 46 days, about 47 days, about 48 days, about 49 days, about 50 days, about 51 days, about 52 days, about 53 days, about 54 days, about 55 days, about 56 days, about 57 days, about 58 days, about 59 days, about 60 days, about 61 days, about 62 days, about 63 days, about 64 days, about 65 days, about 66 days, about 67 days, about 68 days, about 69 days, about 70 days, about 71 days, about 72 days, about 73 days, about 74 days, about 75 days, about 76 days, about 77 days, about 78 days, about 79 days, about 80 days, about 81 days, about 82 days, about 83 days, about 84 days, about 85 days, about 86 days, about 87 days, about 88 days, about 89 days, of about 90 days prior to surgery where the incision is made to the area of skin. In some embodiments of any one of each or any of the above- or below-mentioned embodiments, the composition is administered to the area of skin about 24 hours, about 12 hours, about 8 hours, about 4 hours, about 1 hour, about 30 minutes, about 15 minutes or about 1 minute prior to surgery where the incision is made to the area of skin. In some embodiments, the composition is administered to the area of skin between 7 days and 90 days prior to surgery where the incision is made to the area of skin. In some embodiments, the method further comprises administering the composition again following the incision. In some embodiments, the composition is administered 24 hours, 12 hours, 8 hours, 4 hours or 1 hour following the incision. In some embodiments, the composition is administered between 1 minute and 60 minutes following the incision. In some embodiments, the composition is administered in a volume of about 1 μL to about 100 μL to an area that is about 0.1 cm to about 2 cm along a length of an incision, scrape, scar, cut or wound. In some embodiments, the composition is administered to the area of skin that is about 0.1 cm to about 2 cm away from the incision, scrape, scar, cut or wound. In some embodiments, the composition is administered in a volume of about 1-25 μL, about 25-50 μL, about 50-75 μL, or about 75-100 μL. In some embodiments, the administration of the composition is repeated following surgery. In some embodiments, the patient is known to be prone to keloid type scarring. In some embodiments the volume of composition injected is about 1 μl to 100 μl of composition per square centimeter along the incision or wound length.

In some embodiments, a method of pre-treating skin of a patient to limit scar formation or scar coloration is provided. The method comprises administering a composition comprising tropoelastin to an area of skin that is to receive an incision. The method comprises administering a composition comprising tropoelastin to an area of skin that is to receive an incision. In some embodiments, the tropoelastin is in a monomeric form and is crosslinked with the hyaluronic acid. In some embodiments, the composition comprises about 20 mg/ml, about 30 mg/ml, about 40 mg/ml or about 50 mg/ml recombinant human tropoelastin. In some embodiments, the tropoelastin comprises about 30 mg/ml tropoelastin crosslinked to about 0.5% hyaluronic acid. In some embodiments, the composition comprises 30 mg/ml recombinant human tropoelastin crosslinked with 0.5% hyaluronic acid. In some embodiments, the tropoelastin in the composition is cross-linked to hyaluronic acid, wherein the composition comprises derivatised HA (dHA) or underivatized HA (HA). In some embodiments, the composition is administered by injection at 0.7 to 0.8 cm intervals, along the area of skin that is to receive the incision, along each side of the incision. In some embodiments, the composition is administered between about 1 minute and about 90 days prior to surgery. In some embodiments, the composition is administered 24 hours, 12 hours, 8 hours, 4 hours, 1 hour or 30 minutes prior to surgery. In some embodiments, the composition is administered between 7 days and 28 days prior to surgery. In some embodiments, the method further comprises administering the composition again following the incision. In some embodiments, composition is administered 24 hours, 12 hours, 8 hours, 4 hours or 1 hour following the incision. In some embodiments, the composition is administered between 1 minute and 60 minutes following the incision. In some embodiments, the administration of the composition is repeated following surgery. In some embodiments, the area of incision is a facial area, back, torso or limbs of the patient in need. In some embodiments, the patient is known to be prone to keloid type scarring.

In some embodiments of any one of each or any of the above- or below-mentioned embodiments, the tropoelastin is monomeric, comprises a sequence as set forth in SEQ ID NO: 1, and is cross-linked to the hyaluronic acid.

### Methods

A method is provided for treating and/or preventing scarring in a patient in need thereof by administering a composition comprising tropoelastin to an area of skin comprising a proposed incision site, including, for example, wherein the administering is performed along a length of the proposed incision site about 1 minute to about 90 days prior to receiving an incision.

The composition may be administered to the area of skin between about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, about 31 days, about 32 days, about 33 days, about 34 days, about 35 days, about 36 days, about 37 days, about 38 days, about 39 days, about 40 days, about 41 days, about 42 days, about 43 days, about 44 days, about 45 days, about 46 days, about 47 days, about 48 days, about 49 days, about 50 days, about 51 days, about 52 days, about 53 days, about 54 days, about 55 days, about 56 days, about 57 days, about 58 days, about 59 days, about 60 days, about 61 days, about 62 days, about 63 days, about 64 days, about 65 days, about 66 days, about 67 days, about 68 days, about 69 days, about 70 days, about 71 days, about 72 days, about 73 days, about 74 days, about 75 days, about 76 days, about 77 days, about 78 days, about 79 days, about 80 days, about 81 days, about 82 days, about 83 days, about 84 days, about 85 days, about 86 days, about 87 days, about 88 days, about 89 days, of about 90 days prior to surgery where the incision is made to the area of skin.

The composition used for the methods comprises about 20 mg/ml tropoelastin, about 30 mg/ml tropoelastin, about 40 mg/ml tropoelastin or about 50 mg/ml tropoelastin. In some embodiments, the composition comprises derivatized hyaluronic acid (dHA). In some embodiments, the composition comprises about 0.4% dHA, about 0.5% dHA, about 0.6% dHA, about 0.7% dHA or about 0.8% dHA. In some embodiments, a sterile skin marker will be used to mark 0.7-0.8 cm intervals along each side of proposed incision site. The injections will be administered uniformly along the length of the incision site. In some embodiments, administration further comprises injections administered uniformly at the ends of the incision site.

In some embodiments, wherein the proposed incision is not linear, the tropoelastin composition is injected about 0.5 cm to about 1.5 cm away from the proposed incision site such that there is about 1 μl to 100 μl of composition per square centimeter along the area of the proposed incision site on one side or both sides of the incision. In some embodiments, wherein the proposed incision is not linear, the tropoelastin is not administered along the entire length of the incision and is administered along at least one of the lengths of the incision.

In some embodiments, injections of the composition are made along the site marked for the incision and close to the surface so that the needle may be visualized through the skin margin. In some embodiments, the injection is placed at a depth of about 0.1 cm, about 0.2 cm, about 0.3 cm, about 0.4 cm, about 0.5 cm, about 0.6 cm, about 0.7 cm, about 0.8 cm, about 0.9 cm or about 1 cm below the surface of the skin. In some embodiments, the composition is administered in parallel along the length of the proposed incision site. In some embodiments, the injections along the site of incision are not all equidistant from the site of the proposed incision site.

In some embodiments, the composition is injected in a retrograde fashion such that composition is injected uniformly over the intervals next to the site marked for incision. A slight intradermal bulge containing the injected composition may be visualized as composition is injected and the needle/syringe for administration is simultaneously slowly withdrawn in a retrograde fashion. In some embodiments, the needle is withdrawn parallel or perpendicular to the incision site.

In some embodiments, injections of the composition are made around the wound, wherein the wound is not an incision, such as a fresh abrasion.

In an embodiment, the composition comprising tropoelastin is administered to a patient that is a poor wound healer. In an alternative embodiment, the composition comprising tropoelastin is administered to a patient that is a good wound healer.

Also provided herein are methods to identify a patient as a good wound healer or a poor wound healer including, for example, based on a score (or an average score) attributed to one or more scars (e.g., pre-existing scars) on the patient. Any system used to score a scar is contemplated for use in the methods disclosed herein. For example, a scar may be scored on the Vancouver Scar Scale, Visual Analogue Scale with Scar Ranking, Patient and Observer Scar Assessment Scale, Manchester Scale, or Stony Brook Scar Evaluation Scale (see, e.g., Fearmonti et al., (2011) Plast. Reconstr. Surg. 127(1): 242-247). Such scoring systems may be divided into one or more increments to delineate a poor wound healer from a good wound healer. For example, the Vancouver Scar Scale may be divided such that a score for a scar of 0-6 corresponds to a patient that is a good wound healer and a score of 7-13 corresponds to a patient that is a poor wound healer. Such methods may be combined with any other methods disclosed herein.

In one embodiment, a patient that is a poor wound healer may be identified by scars having one or more of the following characteristics: an obvious sharp border, a raised appearance, presence of a color mismatch, a contour irregularity, and/or a large surface area. Such scars may be scored as a 1 (poor) on a scale of 1 to 4 (i.e., 1, 2, 3, or 4). Additionally, a patient that is a poor wound healer may be identified by scars having one or more of the following characteristics: a scar border that can be discerned, a minimally raised appearance, and/or the presence of a color mismatch. Such scars may be scored as a 2 (average) on a scale of 1 to 4. Conversely, a patient that is a good wound healer may be identified by scars having one or more of the following characteristics: a border that is difficult to discern, not raised, a minimal color mismatch, and/or a texture/wrinkle pattern different to adjacent skin. Such scars may be scored as a 3 (good) on a scale of 1 to 4. Moreover, a patient that is a good wound healer may be identified by scars having one or more of the following characteristics: a border that is not discernible, not raised, no color mismatch, and/or a texture/wrinkle pattern to adjacent skin. Such scars may be scored as a 4 (excellent) on a scale of 1 to 4. In an embodiment, one or more scars (e.g., pre-existing scars) on a patient are scored on a scale of 1 to 4 using the criteria provided above. The scores are then averaged to identify or classify a patient as a good healer or a poor healer. In a further embodiment, a patient is identified or classified as a poor wound healer where the patient has a scar with a score of 1 or 2 (or scars with an average score between 1 and 2.49). Alternatively, a patient is identified or classified as a good wound healer where the patient has a scar with a score of 3 or 4 (or scars with an average score of 2.5 to 4).

In another embodiment, the Vancouver Scar Scale may be used to score a scar. The Vancouver Scar Scale scores a scar from 0 to 13 based on four features of a scar including pigmentation (scored from 0 to 2), vascularity (scored from 0 to 3), pliability (scored from 0 to 5), and height (scored from 0 to 5). Normal pigmentation is scored as a 0 whereas hypopigmentation is scored as a 1 and hyperpigmentation is scored as a 2. Normal vascularity is scored as a 0 whereas pink vascularity is scored as a 1, red vascularity is scored as a 2, and purple vascularity is scored as a 3. Normal pliability is scored as a 0 whereas supple pliability is scored as a 1, yielding pliability is scored as a 2, firm pliability is scored as a 3, banding pliability is scored as a 4, and contracture pliability is scored as a 5. Normal height (flat) is scored as a 0 whereas a height of 0-2 mm is scored as a 1, a height of 2-5 mm is scored as a 2, and a height >5 mm is scored as a 3. The individual scores assigned to pigmentation, vascularity, pliability, and height are then added to determine the Scale score. For example, a scar assigned a pigmentation score of 2, a vascularity score of 3, a pliability score of 2, and a height score of 3 is given a Scale score of 10. In an embodiment, a good wound healer has a Vancouver Scar Scale score of 0, 1, 2, 3, 4, 5, 6, or 7. In another embodiment, a poor wound healer has a Vancouver Scar Scale score of 7, 8, 9, 10, 11, 12, or 13.

In yet another embodiment, the POSAS Patient Scale may be used to score a scar and identify a patient as a good wound healer or a poor wound healer. The POSAS Patient Scale asks a series of seven questions to rate a scar on a scale of 1 to 10 with 1 corresponding to an answer of no, not at all, or as normal skin and 10 corresponding to an answer of yes, very much, or very different. In particular, the POSAS Patient Scale asks i) whether the scar has been painful in the past few weeks, ii) has the scar been itching the past few weeks, iii) is the scar color different from the color of your normal skin at present, iv) is the stiffness of the scar different from your normal skin at present, v) is the thickness of the scar different from your normal skin at present, vi) is the scar more irregular than your normal skin at present, and vii) what is your overall opinion of the scar compared to normal skin. In an embodiment, an average score for each of the eight questions of 1 to 5 indicates that a patient is a good wound healer and an average score of 6 to 10 indicates that a patient is a poor wound healer. In another embodiment, an average score for the scar may be determined for one or more of the eight questions. In still another embodiment, the POSAS Observer Scale may be used to score a scar and identify a patient as a poor wound healer or a good wound healer. The POSAS Observer Scale asks an observer to rate six parameters of a scar on a scale of 1 to 10 with 1 corresponding to an answer of normal skin and 10 corresponding to an answer of worst scar imaginable. The observer is then asked to also provide an overall opinion of the scar on the same scale of 1 to 10. The parameters include vascularity (e.g., the presence of vessels in scar tissue assessed by the amount of redness, tested amount of blood return after blanching with a piece of Plexiglas), pigmentation (e.g., brownish coloration of the scar by pigment), thickness (average distance between the subcutical-dermal border and the epidermal surface of the scar), relief (the extent to which surface irregularities are present), pliability (e.g., suppleness of the scar tested by wrinkling the scar between the thumb and index finger), and surface area (e.g., surface area of the scar in relation to the original wound area). All parameters are preferably compared to normal skin on a comparable anatomic location. In an embodiment, an average score for each of the six parameters of 1 to 5 indicates that a patient is a good wound healer and an average score of 6 to 10 indicates that a patient is a poor wound healer. In another embodiment, an average score for each of the parameters and the overall opinion of 1 to 5 indicates that a patient is a good wound healer and an average score of 6 to 10 indicates that a patient is a poor wound healer.

### Area of Skin to be Treated

In the methods described herein, the composition is administered as an injection directly into a site that is to receive the incision, beneath the site that is to receive the incision, at and/or around the area of skin that is to receive an incision. This may be placed as an intradermal implant, a dermal implant, subdermal implant, within the hypodermis and/or at the subcutaneous level, within 0.1 cm, 0.2 cm, 0.3 cm, 0.4 cm, 0.5 cm, 0.6 cm, 0.7 cm, 0.8 cm, 0.9 cm or 1.0 cm from the wound at any one of the described layers of skin. Without being limiting, the composition may be in the form of a gel or an implant. In some embodiments, the composition may be applied into the area to receive the injection at a dermal area, which is the within the layer of skin in between the epidermis and the subcutaneous tissues. For example, the composition may be applied at the dense irregular connective tissues that comprise this particular region. In some embodiments of any one of each or any of the above- or below-mentioned embodiments, the composition may be applied at the subdermis.

### ILLUSTRATION OF SUBJECT TECHNOLOGY AS CLAUSES

Various examples of aspects of the disclosure are described as numbered clauses (1, 2, 3, etc.) for convenience. These are provided as examples, and do not limit the subject technology.
Clause 1: A method of treating or preventing scarring or scar discoloration in a patient in need thereof, the method comprising: administering a composition comprising tropoelastin to an area of skin comprising an incision site, wherein the administering is performed along a length of the incision site about 1 minute to about 365 days after receiving the incision.
Clause 2: The method of Clause 1, wherein the incision site has a right side and a left side, and wherein the composition is administered along the right side and/or the left side along the length of the incision site.
Clause 3: The method of Clauses 1 or 2, wherein the composition is injected about 0.1 cm, about 0.2 cm, about 0.3 cm, about 0.4 cm, about 0.5 cm, about 0.6 cm, about 0.7 cm, about 0.8 cm about 0.9 cm, about 1 cm, about 1.1 cm, about 1.2 cm, about 1.3 cm, about 1.4 cm or about 1.5 cm away from the incision site.
Clause 4: The method of any one of Clauses 1-3, wherein the incision is from a surgery.
Clause 5: The method of any one of Clauses 1-4, wherein sutures are needed to close the incision, and wherein the administering of the composition is performed prior to and/or after the incision has received sutures.
Clause 6: The method of any one of Clauses 1-5, wherein the administering is performed along the length of the incision wherein the composition is administered every 0.1 cm, 0.2 cm, 0.3 cm, 0.4 cm, 0.5 cm, 0.6 cm, 0.7 cm, 0.8 cm, 0.9 cm or 1.0 cm along the length of the incision site on one or both sides of the incision.
Clause 7: The method of any one of Clauses 1-6, wherein the composition is injected about 0.1 cm, about 0.2 cm, about 0.3 cm, about 0.4 cm, about 0.5 cm, about 0.6 cm, about 0.7 cm, about 0.8 cm about 0.9 cm, about 1 cm, about 1.1 cm, about 1.2 cm, about 1.3 cm, about 1.4 cm or about 1.5 cm away from the incision.
Clause 8: A method of treating skin of a patient to prevent scar formation or scar coloration, the method comprising administering a composition comprising tropoelastin to an area of skin that has received a fresh wound, wherein the fresh wound is an incision or abrasion.
Clause 9: The method of Clause 8, wherein the fresh wound is an abrasion, wherein the composition is administered on an area of skin around the abrasion, wherein the administering is performed around a circumference of the wound at about 0.7 cm to about 0.8 cm intervals along the circumference of the wound, and about 0.1 cm, about 0.2 cm, about 0.3 cm, about 0.4 cm, about 0.5 cm, about 0.6 cm, about 0.7 cm, about 0.8 cm about 0.9 cm, about 1 cm, about 1.1 cm, about 1.2 cm, about 1.3 cm, about 1.4 cm or about 1.5 cm away from the wound.
Clause 10: The method of any one of Clauses 1-9, wherein the administering is performed about 1 minute, about 5 minutes, about 10 minutes, about 20 minutes, about 30 minutes, about 60 minutes, about 120 minutes after the incision or abrasion is made.
Clause 11: The method of Clause 10, wherein the fresh wound is an incision, and wherein the administering is performed along the length of the incision about 1 minute to about 365 after receiving the wound.
Clause 12: The method of any one of Clauses 8-11, wherein the wound is to be sutured, and wherein the administering is performed prior to and/or after suturing of the area of skin that has received the wound.
Clause 13: A method of preventing a keloid in a patient in need, the method comprising administering a composition comprising tropoelastin to an area of skin that has received a wound wherein the administering is performed about 1 minute, about 1 year, about 2 years, about 3 years, about 4 years, about 5 years, about 6 years, about 7 years, about 8 years, about 9 years or about 10 years after the patient has received a wound, and wherein the patient in need is prone to keloids.
Clause 14: The method of Clause 13, wherein the patient is administered the composition repeatedly after a first administration of the composition.
Clause 15: A method of preventing scarring in a patient in need, the method comprising administering a composition comprising tropoelastin to an area of skin comprising a proposed incision site, wherein the administering is performed along a length of the proposed incision site about 1 minute to about 90 days prior to receiving an incision.
Clause 16: The method of Clause 15, wherein the administering is performed every 0.1 cm, 0.2 cm, 0.3 cm, 0.4 cm, 0.5 cm, 0.6 cm, 0.7 cm, 0.8 cm, 0.9 cm or 1.0 cm along the length of the proposed incision site.
Clause 17: The method of Clauses 15 or 16, wherein the proposed incision site has a right side and a left side, and wherein the composition is administered along the right side and/or the left side along the length of the proposed incision site, and wherein the composition is injected about 0.1 cm, about 0.2 cm, about 0.3 cm, about 0.4 cm, about 0.5 cm, about 0.6 cm, about 0.7 cm, about 0.8 cm about 0.9 cm, about 1 cm, about 1.1 cm, about 1.2 cm, about 1.3 cm, about 1.4 cm or about 1.5 cm away from the proposed incision site.
Clause 18: The method of any one of Clauses 15-17, wherein the incision site comprises a first end and a second end, and wherein the composition is administered along the first end and/or the second end of the proposed incision site, and wherein the composition is injected about 0.1 cm, about 0.2 cm, about 0.3 cm, about 0.4 cm, about 0.5 cm, about 0.6 cm, about 0.7 cm, about 0.8 cm about 0.9 cm, about 1 cm, about 1.1 cm, about 1.2 cm, about 1.3 cm, about 1.4 cm or about 1.5 cm away from the proposed incision site at the first end and/or the second end.
Clause 19: The method of Clauses 1-18, wherein the tropoelastin is recombinant human tropoelastin.
Clause 20: The method of Clause 1-19, wherein the composition comprises between 1-100 mg/ml tropoelastin.
Clause 21: The method of any one of Clauses 1-20, wherein the composition comprises about 10 mg/ml, about 20 mg/ml, about 30 mg/ml, about 40 mg/ml or about 50 mg/ml recombinant human tropoelastin.
Clause 22: The method of any one of Clauses 1-21, wherein the composition further comprises hyaluronic acid (HA) in a concentration of about 0.2% to about 10% hyaluronic acid.
Clause 23: The method of any one of Clauses 1-22, wherein the composition comprises about 0.2%, about 0.3%, about 0.4%, about 0.5%, about 0.6%, about 0.7%, about 0.8%, 0.9% or about 1% hyaluronic acid (HA).
Clause 24: The method of any one of Clauses 1-23, wherein the tropoelastin is in a monomeric form.
Clause 25: The method of any one of Clauses 1-24, wherein the tropoelastin is cross-linked to the hyaluronic acid.
Clause 26: The method of any one of Clauses 1-25, wherein the hyaluronic acid is derivatized hyaluronic acid (dHA).
Clause 27: The method of any one of Clauses 1-26, wherein the composition comprises about 30 mg/ml tropoelastin.
Clause 28: The method of any one of Clauses 1-27, wherein the tropoelastin comprises about 30 mg/ml tropoelastin crosslinked to about 0.5% derivatized hyaluronic acid.
Clause 29: The method of any one of Clauses 1-28, wherein the composition further comprises a buffer, wherein the buffer is phosphate buffered saline.
Clause 30: The method of any one of Clauses 1-29, wherein the composition is administered in parallel along the length of the incision site or the proposed incision site.
Clause 31: The method of any one of Clauses 1-30, wherein the composition is administered in a volume of about 1 μL to about 100 μL.
Clause 32: The method of any one of Clauses 1-31, wherein the composition is administered in a volume of about 5 μL to about 20 μL.
Clause 33: The method of any one of Clauses 1-31, wherein the composition is administered in a volume of about 10 μL to about 30 μL.
Clause 34: The method of any one of Clauses 1-31, wherein the composition is administered in a volume of about 20 μL to about 50 μL.
Clause 35: The method of any one of Clauses 1-31, wherein the composition is administered in a volume of about 25-50 μL.
Clause 36: The method of Clause 33, wherein the composition is administered with a 30G needle.
Clause 37: The method of Clause 34, wherein the composition is administered with a 27G needle.
Clause 38: The method of any one of Clauses 1-35, wherein the composition is administered with a 32G, 27G, 25G or 23G needle.
Clause 39: The method of any one of Clauses 1-38, wherein the composition is administered with an injection device.
Clause 40: The method of Clause 39, wherein the injection device is a needle roller ball type system, an automatic injection pen type system or a mesotherapy injection gun type system.
Clause 41: The method of any one of Clauses 1-40, wherein the composition is administered as an intradermal implant, a dermal implant, a subdermal implant, within the hypodermis and/or a subcutaneous area.
Clause 42: The method of any one of Clauses 15-41, wherein the composition is administered as an injection directly into the proposed incision site, beneath the proposed incision site or the area of skin around the proposed incision site, along the length of the proposed incision site.
Clause 43: The method of Clause 42, wherein the composition is administered as an injection directly at the proposed incision site along the length of the proposed incision site.
Clause 44: The method of any one of Clauses 15-43, wherein the composition is administered as an injection beneath the proposed incision site, wherein the composition is injected at about 1 mm, about 2 mm, about 3 mm, about 4 mm, about 5 mm, about 6 mm, about 7 mm, about 8 mm, about 9 mm or about 10 mm, below the proposed incision site.
Clause 45: The method of any one of Clauses 1-44, wherein the composition is injected about 0.1 cm away from the incision site or proposed incision site, on the right side and/or the left side of the injection site or proposed incision site, wherein the composition comprises about 10 mg/ml to about 30 mg/ml recombinant human tropoelastin crosslinked with 0.5% derivatized hyaluronic acid, and wherein the composition is administered in a volume of about 1 μL to about 25 μL.
Clause 46: The method of any one of Clauses 1-44, wherein the composition is injected about 0.5 cm away from the incision site or proposed incision site, on the right side and/or the left side of the proposed incision site, wherein the composition comprises about 30 mg/ml to about 100 mg/ml recombinant human tropoelastin crosslinked with 0.5% derivatized hyaluronic acid, and wherein the composition is administered in a volume of about 25 μL to about 50 μL.
Clause 47: The method of any one of Clauses 1-44, wherein the composition is injected about 1.0 cm away from the incision site or proposed incision site, on the right side and/or the left side of the incision site or proposed incision site, wherein the composition comprises about 30 mg/ml to about 100 mg/ml recombinant human tropoelastin crosslinked with 0.5% derivatized hyaluronic acid, and wherein the composition is administered in a volume of about 50 μL to about 100 μL.
Clause 48: The method of any one of Clauses 1-44, wherein the composition is injected about 1.5 cm away from the incision site or proposed incision site, on the right side and/or the left side of the incision site or proposed incision site, wherein the composition comprises about 30 mg/ml to about 100 mg/ml recombinant human tropoelastin crosslinked with 0.5% derivatized hyaluronic acid, and wherein the composition is administered in a volume of about 75 μL to about 100 μL.
Clause 49: The method any one of Clauses 1-48, wherein the composition is administered by injection at about 0.7 cm to about 0.8 cm intervals along the length of the incision site or proposed incision site.
Clause 50: The method of any one of Clauses 1-49, wherein the composition is administered by injection at 0.7 to 0.8 cm intervals along the length of the incision site or proposed incision site, on the right side and/or the left side of the incision site or proposed incision site.
Clause 51: The method of any one of Clauses 1-50, wherein the incision is performed for surgery.
Clause 52: The method of any one of Clauses 15-51, wherein the composition is administered to the area of skin comprising the incision site or proposed incision site about 90 days prior to receiving the incision.
Clause 53: The method of any one of Clauses 15-52, wherein the composition is administered to the area of skin comprising the proposed incision site about 60 days prior to receiving the incision.
Clause 54: he method of any one of Clauses 15-52, wherein the composition is administered to the area of skin comprising the proposed incision site about 28 days prior to receiving the incision.
Clause 55: The method of any one of Clauses 15-52, wherein the composition is administered to the area of skin comprising the proposed incision site about 1 minute prior to receiving the incision.
Clause 56: The method of any one of Clauses 15-55, wherein the method further comprises making an incision on the proposed incision site and administering the composition following the incision.
Clause 57: The method of Clause 56, wherein the composition is administered about 1 minute, about 5 minutes, about 10 minutes, about 20 minutes, about 30 minutes, about 1 hour, about 2 hours, about 4 hours, about 6 hours, about 12 hours, about 24 hours, about 48 hours, about 72 hours, about 1 week, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months and/or about 12 months following the incision.
Clause 58: The method of any one of Clauses 56 or 57, wherein the method further comprises repeating administration of the composition following the incision.
Clause 59: The method of Clause 58, wherein the composition is administered initially between 1 minute and 60 minutes following the incision.
Clause 60: The method of Clause 58 or 59, wherein repeating administration is performed during a duration of 1 year after making the incision.
Clause 61: The method of any one of Clauses 58-60, wherein the composition is administered repeatedly up to 365 days following the incision.
Clause 62: The method of any one of Clauses 1-61, wherein the patient in need has a skin type on a Fitzpatrick skin type scale of I, II, III, IV, V, or VI.
Clause 63: The method of any one of Clauses 1-62, wherein the area of skin that is to receive the incision is a facial area, back, torso or limbs of the patient in need.
Clause 64: The method of any one of Clauses 1-63, wherein the patient is prone to keloid type scarring.
Clause 65: The method of any one of Clauses 1-64, wherein the surgery is for keloid removal, and the method further prevents keloid recurrence after surgical removal.
Clause 66: The method of any one of Clauses 1-65, wherein the method further prevents formation of scar coloration.
Clause 67: A method of pre-treating skin of a patient to prevent formation of scar and preventing scar discoloration, the method comprising administering a composition comprising tropoelastin to an area of skin comprising a proposed incision site, wherein the administering is performed along a length of the proposed incision site about 1 minute to about 90 days prior to receiving an incision.
Clause 68: The method of Clause 67, wherein the method prevents red, purple, dark brown or white scarring.
Clause 69: A method of performing a surgical procedure, the method comprising:
administering a composition comprising tropoelastin to an area of skin comprising a proposed incision site, wherein the administering is performed about 1 minute to about 90 days along a length of the proposed incision site prior to making an incision; and
making an incision to the area of skin.
Clause 70: The method of Clause 69, further comprising administering the composition following the incision about 1 minute, about 5 minutes, about 10 minutes, about 20 minutes, about 30 minutes, about 1 hour, about 2 hours, about 4 hours, about 6 hours, about 12 hours, about 24 hours, about 48 hours, about 72 hours, about 1 week, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months and/or about 12 months following the incision.
Clause 71: A method of preventing scarring or scar discoloration in a patient in need the method comprising administering a composition comprising tropoelastin to an area of skin comprising an incision site, wherein the administering is performed along a length of the incision site about 1 minute to about 365 days after receiving the incision.
Clause 72: The method of Clause 71, wherein the incision is from surgery.
Clause 73: The method of Clauses 71 of 72, wherein sutures are needed to close the incision, and wherein the administering of the composition is performed prior to and/or after the incision has received sutures.
Clause 74: The method of any one of Clauses 71-73, wherein the administering is performed along a length of the incision wherein the composition is administered every 0.1 cm, 0.2 cm, 0.3 cm, 0.4 cm, 0.5 cm, 0.6 cm, 0.7 cm, 0.8 cm, 0.9 cm or 1.0 cm along the length of the incision site on one or both sides of the incision.
Clause 75: The method of any one of Clauses 71-74, wherein the composition is injected about 0.1 cm, about 0.2 cm, about 0.3 cm, about 0.4 cm, about 0.5 cm, about 0.6 cm, about 0.7 cm, about 0.8 cm about 0.9 cm, about 1 cm, about 1.1 cm, about 1.2 cm, about 1.3 cm, about 1.4 cm or about 1.5 cm away from the incision.
Clause 76: A method of treating skin of a patient to prevent scar formation or scar coloration, the method comprising administering a composition comprising tropoelastin to an area of skin that has received a fresh wound, wherein the fresh wound is an incision or abrasion.
Clause 77: The method of Clause 76, wherein the fresh wound is an abrasion, wherein the composition is administered on an area of skin around the abrasion, wherein the administering is performed around a circumference of the wound at about 0.7 cm to about 0.8 cm intervals along the circumference of the wound, and about 0.1 cm, about 0.2 cm, about 0.3 cm, about 0.4 cm, about 0.5 cm, about 0.6 cm, about 0.7 cm, about 0.8 cm about 0.9 cm, about 1 cm, about 1.1 cm, about 1.2 cm, about 1.3 cm, about 1.4 cm or about 1.5 cm away from the wound.
Clause 78: The method of Clauses 76 or 77, wherein the administering is performed about 1 minute, about 5 minutes, about 10 minutes, about 20 minutes, about 30 minutes, about 60 minutes, about 120 minutes after the incision or abrasion is made.
Clause 79: The method of Clause 76, wherein the fresh wound is an incision, and wherein the administering is performed along a length of the incision about 1 minute to about 365 after receiving the wound.
Clause 80: The method of any one of Clauses 76-79, wherein the wound is to be sutured, and wherein the administering is performed prior to and/or after suturing of the area of skin that has received the wound.
Clause 81: A method of preventing a keloid in a patient in need, the method comprising administering a composition comprising tropoelastin to an area of skin that has received a wound wherein the administering is performed about 1 minute, about 1 year, about 2 years, about 3 years, about 4 years, about 5 years, about 6 years, about 7 years, about 8 years, about 9 years or about 10 years after the patient has received a wound, and wherein the patient in need is prone to keloids.
Clause 82: The method of Clause 81, wherein the patient is administered the composition repeatedly after a first administration of the composition.
Clause 83: A method of incising an area of skin on a subject, the method comprising:
making an incision to the area of skin on the subject; and
administering a composition comprising tropoelastin to the area of skin.
Clause 84: A method for preventing scarring in a subject in need thereof, the method comprising:
administering a composition comprising tropoelastin along a length of an incision site, wherein the subject is a poor wound healer.
Clause 85: A method for preventing scarring in a subject having or receiving an incision, the method comprising:
identifying the subject as a poor wound healer or a good wound healer; and
administering a composition comprising tropoelastin along a length of the incision where the subject is identified as a poor wound healer.

### EXAMPLES

### Example 1: RhTE Injection Following Incision of Skin for Preventing Scarring

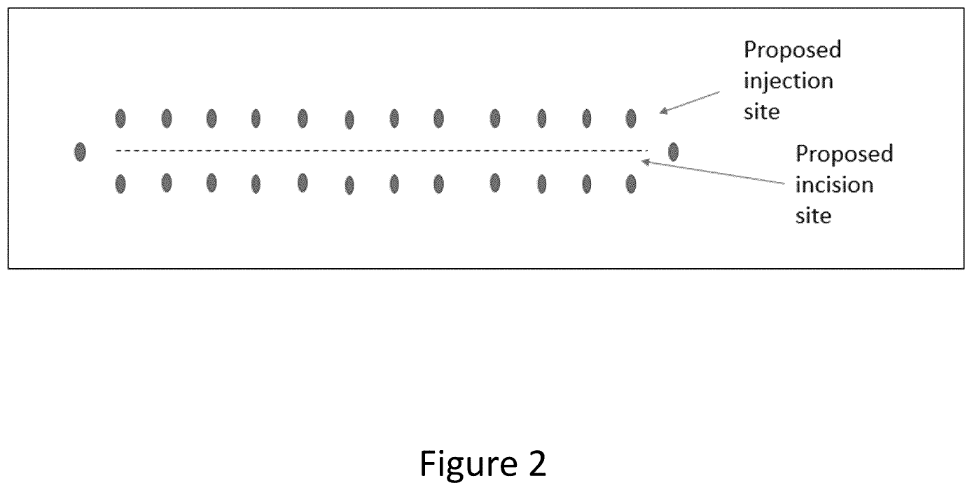

A sterile skin marker was used to draw a site for the incision. The marker is also used to mark 0.7-0.8 cm intervals along each side of the proposed incision site that will have the incision or surgical wound (e.g., using a 1 mL BD syringe prefilled with approximately 0.7 mL of rhTE product that is stored at 2-8° C. until use, which must occur within 30 minutes of removing the syringe from storage, with a 27G×½″ needle with Luer lock). These dot marks will be used to guide injection placement as described below. (FIG. 2)

There will be 12 pigs used for the test, in which each pig will receive two incisions on the back. The wounds are separated by 12 inches. The control wound receives an injection of PBS. The test wound receives the rhTE product. Assuming the 27G needle is ˜1 cm (½″) in length, intradermal injections will be made at 0.7-0.8 cm intervals using the technique as described in Example 1. After incision, the needle is inserted directly into the dermis at an angle of approximately 30°. Insertion will be as far as the needle can go with the bevel facing upwards; the needle insertion trajectory should be parallel with the skin surface and close to the surface so that the needle shape can be clearly visualized through the skin.

rhTE or PBS control will be injected in a retrograde fashion such that 30-40 μl is injected uniformly over 0.7-0.8 cm. A slight intradermal bulge containing the injected rhTE will be visualized as rhTE is injected and the needle/syringe is simultaneously slowly withdrawn in a retrograde fashion.

The next injection will be performed in the same manner ensuring that the next “thread” of material begins where the last thread ends. Separate injections will be required on each side of the site where the incision or surgical wound was made. The injections will be performed after making the incision about 1 minute after making the incision.

After surgery, additional injections may be administered following the incision. The composition may be administered again 1 week, 48 hours, 24 hours, 12 hours following the incision or immediately after the incision. It is expected that the incisions that receive the injections will have less pronounced scarring as compared to the control injections.

The composition may be administered for about 1 minute, about 5 minutes, about 10 minutes, about 20 minutes, about 30 minutes, about 1 hour, about 2 hours, about 4 hours, about 6 hours, about 12 hours, about 24 hours, about 48 hours, about 72 hours, about 1 week, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months and/or about 12 months following the incision.

The composition may be administered until there is no sign of scarring.

### Example 2: RhTE Injection Technique for Preventing Scarring after Incision

A sterile skin marker was used to mark 0.7-0.8 cm intervals along each side of proposed incision site. These dot marks were used to guide injection placement as described below.

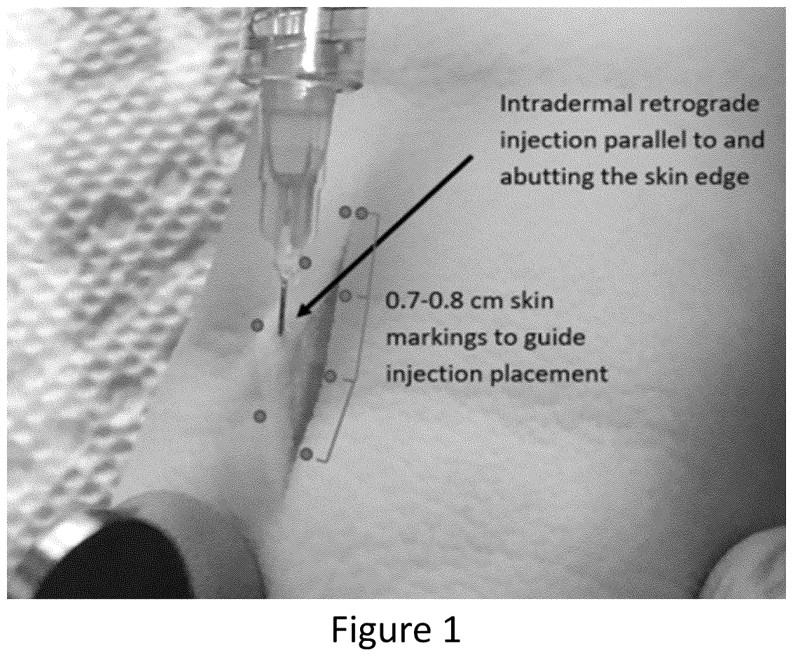

Intradermal injections were made at 0.7-0.8 cm intervals in the following manner (see FIG. 1; e.g., using a 1 mL BD syringe prefilled with approximately 0.7 mL of rhTE product that is stored at 2-8° C. until use, which occurred within 30 minutes of removing the syringe from storage, with a 27G×½ needle with Luer lock).

Injections were made parallel to the site marked for the incision and close to the surface so that the needle was visualized through the skin margin. rhTE was injected in a retrograde fashion such that 30-40 pls were injected uniformly over 0.7-0.8 cm. A slight intradermal bulge containing the injected rhTE was visualized as rhTE was injected and the needle/syringe was simultaneously slowly withdrawn in a retrograde fashion. The next injections were performed in the same manner ensuring that the next “thread” of material began where the last thread ended. Four separate injections were placed on each side of a proposed 3 cm incisional wound site such that approximately 300 μl of rhTE was injected in total.

### Example 3: RhTE Injection Prior to Incision for Preventing Scarring

A sterile skin marker will be used to draw a site for the incision. The marker is also used to mark 0.7-0.8 cm intervals along each side of the proposed incision site that will have the incision or surgical wound (e.g., using a 1 mL BD syringe prefilled with approximately 0.7 mL of rhTE product that is stored at 2-8° C. until use, which must occur within 30 minutes of removing the syringe from storage, with a 27G×½″ needle with Luer lock). These dot marks will be used to guide injection placement as described below. (FIG. 2)

There will be 12 pigs used for the test, in which each pig will receive two incisions on the back. The wounds are separated by 12 inches. The control wound receives an injection of PBS. The test wound receives the rhTE product.

Assuming the 27G needle is ˜1 cm (½″) in length, intradermal injections will be made at 0.7-0.8 cm intervals using the technique as described in Example 1. The needle will be inserted directly into the dermis at an angle of approximately 30°. Insertion will be as far as the needle can go with the bevel facing upwards; the needle insertion trajectory should be parallel with the skin surface and close to the surface so that the needle shape can be clearly visualized through the skin.

rhTE or PBS control will be injected in a retrograde fashion such that 30-40 μl is injected uniformly over 0.7-0.8 cm. A slight intradermal bulge containing the injected rhTE will be visualized as rhTE is injected and the needle/syringe is simultaneously slowly withdrawn in a retrograde fashion.

The next injection will be performed in the same manner ensuring that the next “thread” of material begins where the last thread ends. Separate injections will be required on each side of site where the incision or surgical wound will be made. The injections will be performed prior making the incision about 48 hours prior to making the incision.

After surgery, additional injections may be administered following the incision. The composition may be administered again 1 week, 48 hours, 24 hours, 12 hours following the incision or immediately after the incision. It is expected that the incisions that receive the injections will have less pronounced scarring as compared to the control injections.

The composition may be administered for about 1 minute, about 5 minutes, about 10 minutes, about 20 minutes, about 30 minutes, about 1 hour, about 2 hours, about 4 hours, about 6 hours, about 12 hours, about 24 hours, about 48 hours, about 72 hours, about 1 week, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months and/or about 12 months following the incision.

### Example 4: Biopsy and Wound Healing

Wound healing was examined in a randomized, within-subject, saline control blinded study in women with striae distensae (SD) alba in women using a treatment of Composition A (30 mg/mL+0.5% dHA) or Composition B (40 mg/mL+0.4% dHA).

The study was performed for 18 months with a follow up. The women ranged in age from 30-55 years, were on the Fitzpatrick scale of II, III or IV. The skin studied was on the abdominal area, hip area or thigh area.

Two formulations were used in the testing, Composition A (30 mg/mL+0.5% dHA) and Composition B (40 mg/mL+0.4% dHA). 3 intradermal injections were performed at D0, D28 and D56. At D84 a biopsy (i.e. one month after last Rx) was performed. The scars were photographed using 2D and 3D photography and the physical appearance of the scar was assessed.

The method employed was to create and validate the biopsy wound healing rating scale. Scar healing was assayed using Composition A and B with the control (saline injection) to confirm if the tropoelastin compositions improve the quality of the biopsy site wound healing.

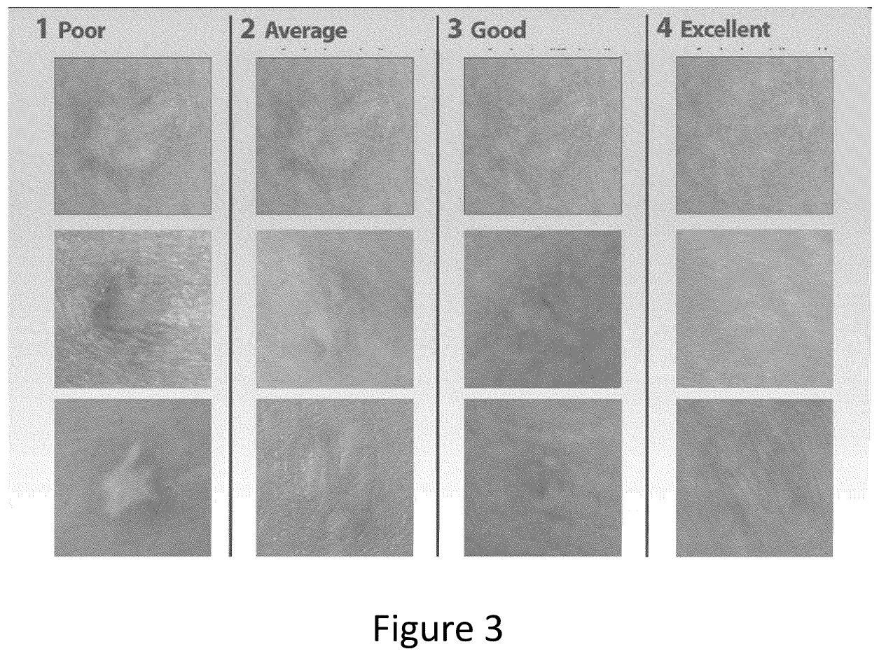

A scale was developed to guide rating of images of the scar healing. (FIG. 3). The four categories of healing are shown in FIG. 3. Photographs of the subjects were taken at day 504 and day 336 were used to develop and validate a scoring system for wound healing. This involved various steps.
Step 1 (review 1): 31 unpaired day 504 photographs were graded (according to the four point scale below) by four reviewers to select 2-3 photographs to represent each grade and further develop the score by providing descriptions and sample photographs.
Step 2 (review 2 round 1): 34 unpaired D336 images were graded by several reviewers using the scale developed in step 1. This step was used to further develop and validate the system (descriptions and sample photographs) and to determine consistency of grading amongst different reviewers.
Step 3 (review 2 round 2): The 34 photographs used in step 2 were re-randomized and regraded by the same reviewers in step 2. This step provides an assessment of reliability by asking the same reviewers to score the same photographs on two separate occasions, separated by at least 14 days.
Step 4: 15 paired D336 images were used. These represented two photographs for the same subject, one being from a treated (Composition A or B) and the other an untreated (saline). The reviewer was asked to determine which photograph represented better healing; i.e. they needed to provide a ranking. The reviewers were asked to use the scale developed in the previous steps to make a decision.

The grading scale for step 1 was to grade the wound on a 4 point scale: 1—Poor; 2—Average; 3—Good; and 4—Excellent. The criteria of the review was performed as described using the criterion defined by Landis and Koch (1977). Ratings of the images of the scarring is shown in Table 1 below:

| | | Tropoelastin | |
|---|---|---|---|
| Parameter | Saline | composition | P value |
| Average score (D336) | 2.2 | 2.5 | |
| Difference (Saline) @D336 | 0.32 | | 0.004 |
| Average score (D504) | 2.3 | 2.8 | |

| | | |
|---|---|---|
| Difference (ELAPR-saline) | 0.6 | <0.0001 |
| @D504 | | |
| Composition A difference | 0.38 | 0.0022 |
| (saline) @ D336 | | |
| Composition B difference | 0.25 | 0.004 |
| (saline) @ D336 | | |
| Composition A difference | 0.8 | 0.035 |
| (saline) @ D504 | | |
| Composition B difference | 0.5 | 0.0355 |
| (saline) @ D504 | | |

As shown, the compositions comprising tropoelastin perform better than saline (statistically significant. Composition A was shown to perform better than Composition B (not statistically significant). This difference was shown to increase over time (D504>D336).

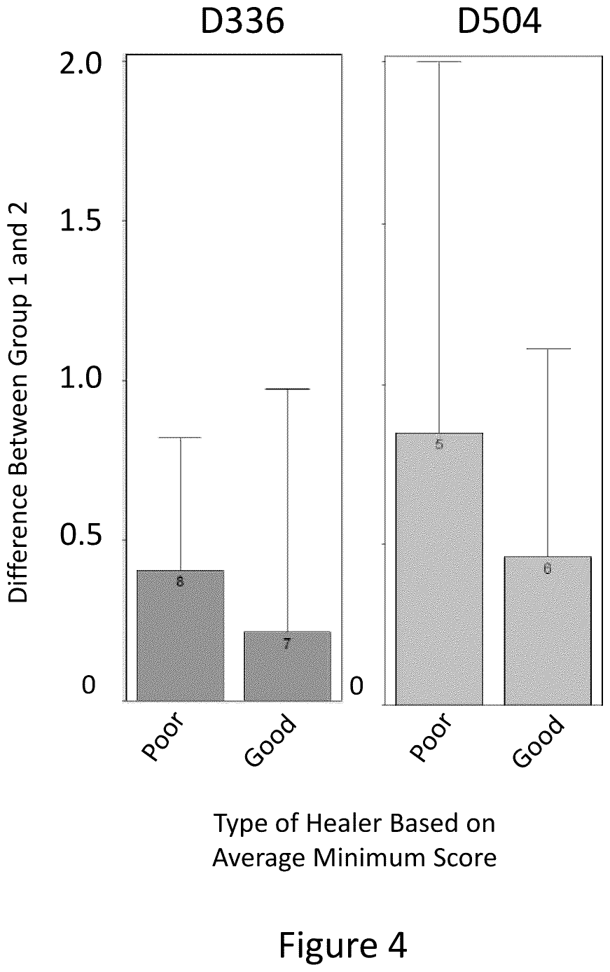

As shown from the results between the groups treated with Composition A, B or control (saline), compositions comprising tropoelastin may do better in poorer healing wounds. (FIG. 4). In the ratings of the images, it was shown that Composition A performed better than Composition B (data not shown). Further, the data also suggest that there is a decrease in the effect of the compositions comprising tropoelastin when the subject had skin that had in increase in grade on the Fitzpatrick scale, although this was minimal when using Composition B as compared to Composition A.

With respect to the use of plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those of skill within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.).

It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations).

Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.

In addition, where features or aspects of the disclosure are described in terms of Markush groups, those skilled in the art will recognize that the disclosure is also thereby described in terms of any individual member or subgroup of members of the Markush group.

Any of the features of an embodiment of the first through ninth aspects is applicable to all aspects and embodiments identified herein. Moreover, any of the features of an embodiment of the first through ninth aspects is independently combinable, partly or wholly with other embodiments described herein in any way, e.g., one, two, or three or more embodiments may be combinable in whole or in part. Further, any of the features of an embodiment of the first through sixth aspects may be made optional to other aspects or embodiments.

1. A method of preventing scarring in an area of skin in a subject in need thereof, the method comprising administering a composition comprising tropoelastin to the area of skin that comprises an incision site after the skin has been incised, wherein the composition is administered along a length of the incision site.

2. The method of claim 1, wherein the composition is administered about 1 minute, about 5 minutes, about 10 minutes, about 20 minutes, about 30 minutes, about 1 hour, about 2 hours, about 4 hours, about 6 hours, about 12 hours, about 24 hours, about 48 hours, about 72 hours, about 1 week, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months and/or about 12 months after the skin has been incised.

3. The method of claim 1, wherein the incision site has a right side, a left side, first end and second end, and wherein the composition is administered along the length of the incision site and along the right side and/or the left side of the incision site, and at the first end and/or the second end, and wherein the composition is injected about 0.1 cm, about 0.2 cm, about 0.3 cm, about 0.4 cm, about 0.5 cm, about 0.6 cm, about 0.7 cm, about 0.8 cm about 0.9 cm, about 1 cm, about 1.1 cm, about 1.2 cm, about 1.3 cm, about 1.4 cm or about 1.5 cm away from the incision site.

4. The method of claim 1, wherein the composition comprises between about 1-100 mg/ml tropoelastin.

5. The method of claim 1, wherein the composition further comprises about 0.2% to about 10% hyaluronic acid (HA).

6. The method of claim 1, wherein the tropoelastin is in a monomeric form.

7. The method of claim 5, wherein the tropoelastin is cross-linked to the HA.

8. The method of claim 1, wherein the composition comprises about 30 mg/ml tropoelastin crosslinked to about 0.5% derivatized hyaluronic acid.

9. The method of claim 1, wherein the composition is administered in a volume of about 1 μL to about 100 μL.

10. The method of claim 1, wherein the composition is administered with a 32G, 27G, 25G or 23G needle.

11. (canceled)

12. The method of claim 1, wherein the composition is administered as an injection directly into the incision site, beneath the incision site or the area of skin around the incision site, and/or along a length of the incision site.

13. The method of claim 1, wherein the composition is administered as an injection beneath the incision site, and wherein the composition is injected at about 1 mm, about 2 mm, about 3 mm, about 4 mm, about 5 mm, about 6 mm, about 7 mm, about 8 mm, about 9 mm or about 10 mm, below the incision site.

14. The method of claim 1, wherein the composition is injected about 0.1 cm away from the incision site, on the right side and/or the left side of the incision site, wherein the composition comprises about 10 mg/ml to about 30 mg/ml recombinant human tropoelastin crosslinked with 0.5% derivatized hyaluronic acid, and wherein the composition is administered in a volume of about 1 μL to about 25 μL.

15. The method of claim 1, wherein the composition is injected about 0.5 cm away from the incision site, on the right side and/or the left side of the incision site, wherein the composition comprises about 30 mg/ml to about 100 mg/ml recombinant human tropoelastin crosslinked with 0.5% derivatized hyaluronic acid, and wherein the composition is administered in a volume of about 25 μL to about 50 μL; or
wherein the composition is injected about 1.0 cm away from the incision site, on the right side and/or the left side of the incision site, wherein the composition comprises about 30 mg/ml to about 100 mg/ml recombinant human tropoelastin crosslinked with 0.5% derivatized hyaluronic acid, and wherein the composition is administered in a volume of about 50 μL to about 100 μL; or
wherein the composition is injected about 1.5 cm away from the incision site, on the right side and/or the left side of the incision site, wherein the composition comprises about 30 mg/ml to about 100 mg/ml recombinant human tropoelastin crosslinked with 0.5% derivatized hyaluronic acid, and wherein the composition is administered in a volume of about 75 μL to about 100 μL.

16. The method of claim 1, wherein the composition is administered by injection at about 0.7 cm to about 0.8 cm intervals along a length of the incision site.

17. (canceled)

18. The method of claim 1, wherein the composition is administered to the area of skin comprising the incision site about 1 minute, about 90 days, about 60 days, or about 28 days after receiving the incision.

19.-21. (canceled)

22. The method of claim 1, wherein the subject in need thereof is prone to keloid type scarring, and wherein the method minimizes or prevents keloid scarring.

23.-24. (canceled)

25. A method of preventing scarring or scar discoloration in a subject in need thereof, the method comprising: administering a composition comprising tropoelastin to an area of skin comprising an incision site, wherein composition is administered along a length of the incision site about 1 minute to about 365 days after receiving the incision.

26. (canceled)

27. A method of treating an area of skin of a subject in need thereof to prevent scar formation or scar coloration, the method comprising: administering a composition comprising tropoelastin to the area of skin that has received a fresh wound, wherein the fresh wound is an incision or abrasion.

28. (canceled)

29. A method of preventing a keloid in a subject in need thereof, the method comprising: administering a composition comprising tropoelastin to an area of skin that has received a wound. wherein the composition is administered about 1 minute, about 1 year, about 2 years, about 3 years, about 4 years, about 5 years, about 6 years, about 7 years, about 8 years, about 9 years or about 10 years after the subject has received a wound, and wherein the subject in need thereof is prone to keloids.

30. (canceled)


SEQUENCE LISTING (15 sequences)

SEQ ID NO: 1 - protein, 698 aa, synthetic construct
  REGION 1..698: note = Protein 1
GGVPGAIPGGVPGGVFYPGAGLGALGGGALGPGGKPLKPVPGGLAGAGLGAGLGAFPAVTFPGALVPGGVADAAAAYKAAKAGAGLGGVPGVGGLGVSAGAVVPQPGAGVKPGKVPGVGLPGVYPGGVLPGARFPGVGVLPGVPTGAGVKPKAPGVGGAFAGIPGVGPFGGPQPGVPLGYPIKAPKLPGGYGLPYTTGKLPYGYGPGGVAGAAGKAGYPTGTGVGPQAAAAAAAKAAAKFGAGAAGVLPGVGGAGVPGVPGAIPGIGGIAGVGTPAAAAAAAAAAKAAKYGAAAGLVPGGPGFGPGVVGVPGAGVPGVGVPGAGIPVVPGAGIPGAAVPGVVSPEAAAKAAAKAAKYGARPGVGVGGIPTYGVGAGGFPGFGVGVGGIPGVAGVPSVGGVPGVGGVPGVGISPEAQAAAAAKAAKYGVGTPAAAAAKAAAKAAQFGLVPGVGVAPGVGVAPGVGVAPGVGLAPGVGVAPGVGVAPGVGVAPGIGPGGVAAAAKSAAKVAAKAQLRAAAGLGAGIPGLGVGVGVPGLGVGAGVPGLGVGAGVPGFGAVPGALAAAKAAKYGAAVPGVLGGLGALGGVGIPGGVVGAGPAAAAAAAKAAAKAAQFGLVGAAGLGGLGVGGLGVPGVGGLGGIPPAAAAKAAKYGAAGLGGVLGGAGQFPLGGVAARPGFGLSPIFPGGACLGKACGRKRK

SEQ ID NO: 2 - protein, 733 aa, synthetic construct
  REGION 1..733: note = Protein 2
SMGGVPGAIPGGVPGGVFYPGAGLGALGGGALGPGGKPLKPVPGGLAGAGLGAGLGAFPAVTFPGALVPGGVADAAAAYKAAKAGAGLGGVPGVGGLGVSAGAVVPQPGAGVKPGKVPGVGLPGVYPGGVLPGARFPGVGVLPGVPTGAGVKPKAPGVGGAFAGIPGVGPFGGPQPGVPLGYPIKAPKLPGGYGLPYTTGKLPYGYGPGGVAGAAGKAGYPTGTGVGPQAAAAAAAKAAAKFGAGAAGVLPGVGGAGVPGVPGAIPGIGGIAGVGTPAAAAAAAAAAKAAKYGAAAGLVPGGPGFGPGVVGVPGAGVPGVGVPGAGIPVVPGAGIPGAAVPGVVSPEAAAKAAAKAAKYGARPGVGVGGIPTYGVGAGGFPGFGVGVGGIPGVAGVPSVGGVPGVGGVPGVGISPEAQAAAAAKAAKYGVGTPAAAAAKAAAKAAQFGLVPGVGVAPGVGVAPGVGVAPGVGLAPGVGVAPGVGVAPGVGVAPGIGPGGVAAAAKSAAKVAAKAQLRAAAGLGAGIPGLGVGVGVPGLGVGAGVPGLGVGAGVPGFGAGADEGVRRSLSPELREGDPSSSQHLPSTPSSPRVPGALAAAKAAKYGAAVPGVLGGLGALGGVGIPGGVVGAGPAAAAAAAKAAAKAAQFGLVGAAGLGGLGVGGLGVPGVGGLGGIPPAAAAKAAKYGAAGLGGVLGGAGQFPLGGVAARPGFGLSPIFPGGACLGKACGRKRK

SEQ ID NO: 3 - protein, 698 aa, synthetic construct; identical to SEQ ID NO: 1
  REGION 1..698: note = Protein 3

SEQ ID NO: 4 - protein, 660 aa, synthetic construct
  REGION 1..660: note = Protein 4
GGVPGAVPGGVPGGVFYPGAGFGAVPGGVADAAAAYKAAKAGAGLGGVPGVGGLGVSAGAVVPQPGAGVKPGKVPGVGLPGVYPGFGAVPGARFPGVGVLPGVPTGAGVKPKAPGVGGAFAGIPGVGPFGGPQPGVPLGYPIKAPKLPGGYGLPYTTGKLPYGYGPGGVAGAAGKAGYPTGTGVGPQAAAAAAAKAAAKFGAGAAGFGAVPGVGGAGVPGVPGAIPGIGGIAGVGTPAAAAAAAAAAKAAKYGAAAGLVPGGPGFGPGVVGVPGFGAVPGVGVPGAGIPVVPGAGIPGAAGFGAVSPEAAAKAAAKAAKYGARPGVGVGGIPTYGVGAGGFPGFGVGVGGIPGVAGVPSVGGVPGVGGVPGVGISPEAQAAAAAKAAKYGVGTPAAAAAKAAAKAAQFGLVPGVGVAPGVGVAPGVGVAPGVGLAPGVGVAPGVGVAPGVGVAPGIGPGGVAAAAKSAAKVAAKAQLRAAAGLGAGIPGLGVGVGVPGLGVGAGVPGLGVGAGVPGFGAVPGALAAAKAAKYGAVPGVLGGLGALGGVGIPGGVVGAGPAAAAAAAKAAAKAAQFGLVGAAGLGGLGVGGLGVPGVGGLGGIPPAAAAKAAKYGAAGLGGVLGGAGQFPLGGVAARPGFGLSPIFPGGACLGKACGRKRK

SEQ ID NO: 5 - protein, 732 aa, synthetic construct
  REGION 1..732: note = Protein 5
SMGGVPGAIPGGVPGGVFYPGAGLGALGGGALGPGGKPLKPVPGGLAGAGLGAGLGAFPAVTFPGALVPGGVADAAAAYKAAKAGAGLGGVPGVGGLGVSAGAVVPQPGAGVKPGKVPGVGLPGVYPGGVLPGARFPGVGVLPGVPTGAGVKPKAPGVGGAFAGIPGVGPFGGPQPGVPLGYPIKAPKLPGGYGLPYTTGKLPYGYGPGGVAGAAGKAGYPTGTGVGPQAAAAAAAKAAAKFGAGAAGVLPGVGGAGVPGVPGAIPGIGGIAGVGTPAAAAAAAAAAKAAKYGAAAGLVPGGPGFGPGVVGVPGAGVPGVGVPGAGIPVVPGAGIPGAAVPGVVSPEAAAKAAAKAAKYGARPGVGVGGIPTYGVGAGGFPGFGVGVGGIPGVAGVPSVGGVPGVGGVPGVGISPEAQAAAAAKAAKYGVGTPAAAAAKAAAKAAQFGLVPGVGVAPGVGVAPGVGVAPGVGLAPGVGVAPGVGVAPGVGVAPGIGPGGVAAAAKSAAKVAAKAQLRAAAGLGAGIPGLGVGVGVPGLGVGAGVPGLGVGAGVPGFGAGADEGVRRSLSPELREGDPSSSQHLPSTPSSPRVPGALAAAKAAKYGAAVPGVLGGLGALGVGIPGGVVGAGPAAAAAAAKAAAKAAQFGLVGAAGLGGLGVGGLGVPGVGGLGGIPPAAAAKAAKYGAAGLGGVLGGAGQFPLGGVAARPGFGLSPIFPGGACLGKACGRKRK

SEQ ID NO: 6 - protein, 698 aa, synthetic construct; identical to SEQ ID NO: 1
  REGION 1..698: note = Protein 6

SEQ ID NO: 7 - protein, 660 aa, synthetic construct
  REGION 1..660: note = Protein 7
MGGVPGAVPGGVPGGVFYPGAGFGAVPGGVADAAAAYKAAKAGAGLGGVPGVGGLGVSAGAVVPQPGAGVKPGKVPGVGLPGVYPGFGAVPGARFPGVGVLPGVPTGAGVKPKAPGVGGAFAGIPGVGPFGGPQPGVPLGYPIKAPKLPGGYGLPYTTGKLPYGYGPGGVAAAGKAGYPTGTGVGPQAAAAAAAKAAAKFGAGAAGFGAVPGVGGAGVPGVPGAIPGIGGIAGVGTPAAAAAAAAAAKAAKYGAAAGLVPGGPGFGPGVVGVPGFGAVPGVGVPGAGIPVVPGAGIPGAAGFGAVSPEAAAKAAAKAAKYGARPGVGVGGIPTYGVGAGFFPGFGVGVGGIPGVAGVPSVGGVPGVGGVPGVGISPEAQAAAAAKAAKYGVGTPAAAAAKAAAKAAQFGLVPGVGVAPGVGVAPGVGVAPGVGLAPGVGVAPGVGVAPGVGVAPGIGPGGVAAAAKSAAKVAAKAQLRAAAGLGAGIPGLGVGVGVPGLGVGAGVPGLGVGAGVPGFGAVPGALAAAKAAKYGAVPGVLGGLGALGGVGIPGGVVGAGPAAAAAAAKAAAKAAQFGLVGAAGLGGLGVGGLGVPGVGGLGGIPPAAAAKAAKYGAAGLGGVLGGAGQFPLGGVAARPGFGLSPIFPGGACLGKACGRKRK

SEQ ID NO: 8 - protein, 147 aa, synthetic construct
  REGION 1..147: note = Protein 8
SAMGGVPGALAAAKAAKYGAAVPGVLGGLGALGGVGIPGGVVGAGPAAAAAAAKAAAKAAQFGLVGAAGLGGLGVGGLGVPGVGGLGGIPPAAAAKAAKYGAAGLGGVLGGAGQFPLGGVAARPGFGLSPIFPGGACLGKACGRKRK

SEQ ID NO: 9 - protein, 200 aa, synthetic construct
  REGION 1..200: note = Protein 9
SAMGALVGLGVPGLGVGAGVPGFGAGADEGVRRSLSPELREGDPSSSQHLPSTPSSPRVPGALAAAKAAKYGAAVPGVLGGLGALGGVGIPGGVVGAGPAAAAAAAKAAAKAAQFGLVGAAGLGGLGVGGLGVPGVGGLGGIPPAAAAKAAKYGAAGLGGVLGGAGQFPLGGVAARPGFGLSPIFPGGACLGKACGRKRK

SEQ ID NO: 10 - protein, 56 aa, synthetic construct
  REGION 1..56: note = Protein 10
GIPPAAAAKAAKYGAAGLGGVLGGAGQFPLGGVAARPGFGLSPIFPGGACLGKACG

SEQ ID NO: 11 - protein, 47 aa, synthetic construct
  REGION 1..47: note = Protein 11
GAAGLGGVLGGAGQFPLGGVAARPGFGLSPIFPGGACLGKACGRKRK

SEQ ID NO: 12 - protein, 34 aa, synthetic construct
  REGION 1..34: note = Protein 12
GADEGVRRSLSPELREGDPSSSQHLPSTPSSPRV

SEQ ID NO: 13 - protein, 34 aa, synthetic construct
  REGION 1..34: note = Protein 13
GADEGVRRSLSPELREGDPSSSQHLPSTPSSPRF

SEQ ID NO: 14 - protein, 216 aa, synthetic construct
  REGION 1..216: note = Protein 14
AAAGLGAGIPGLGVGVGVPGLGVGAGVPGLGVGAGVPGFGAGADEGVRRSLSPELREGDPSSSQHLPSTPSSPRVPGALAAAKAAKYGAAVPGVLGGLGALGGVGIPGGVVGAGPAAAAAAAKAAAKAAQFGLVGAAGLGGLGVGGLGVPGVGGLGGIPPAAAAKAAKYGAAGLGGVLGGAGQFPLGGVAARPGFGLSPIFPGGACLGKACGRKRK

SEQ ID NO: 15 - protein, 183 aa, synthetic construct
  REGION 1..183: note = Protein 15
AAAGLGAGIPGLGVGVGVPGLGVGAGVPGLGVGAGVPGFGAVPGALAAAKAAKYGAAVPGVLGGLGALGGVGIPGGVVGAGPAAAAAAAKAAAKAAQFGLVGAAGLGGLGVGGLGVPGVGGLGGIPPAAAAKAAKYGAAGLGGVLGGAGQFPLGGVAARPGFGLSPIFPGGACLGKACGRKRK